(19) United States
(12) Patent Application Publication
(10) Pub. No.: US 2026/0351539 A1
(43) Pub. Date: Nov. 5, 2026

(54) Sequencing of Morpholino Oligomers Using Electron Capture Dissociation

(71) Applicant: DH Technologies Development Pte. Ltd., Singapore (SG)

(72) Inventors: Takashi BABA, Richmond Hill (CA); Kaoru KARASAWA, Tokyo (JP)

(21) Appl. No.: 19/145,993

(22) PCT Filed: Jan. 9, 2024
(86) PCT No.: PCT/IB2024/050202

§ 371 Date: Jul. 7, 2025

Related U.S. Application Data
(60) Provisional application No. 63/479,024, filed on Jan. 9, 2023.

(51) Int. Cl. H01J 49/00; C12Q 1/6872

(52) U.S. Cl. CPC H01J 49/0054; C12Q 1/6872; H01J 49/0031

(57) ABSTRACT

Methods of identifying subjects having complement-related disorders, or at risk of such disorders, are disclosed. Also disclosed are methods for selecting subjects for treatment with complement-targeted therapies, and methods of treatment of subjects with such therapies.

### TECHNICAL FIELD

The teachings herein relate to methods and systems for sequencing a morpholino oligomer using mass spectrometry. More particularly, the teachings herein relate to systems and methods for sequencing a morpholino oligomer using electron-based dissociation mass spectrometry.

The systems and methods herein can be performed in conjunction with a processor, a controller, or a computer system, such as the computer system of FIG. 1B.

### BACKGROUND

### Sequencing Morpholino Oligonucleotides

Morpholino oligonucleotides are synthetic oligonucleotides. Morpholino oligonucleotides are essentially deoxyribonucleic acid (DNA) connected to a backbone of methylenemorpholine rings linked through phosphorodiamidate groups. These oligonucleotides were designed to prevent the translation of messenger ribonucleic acid (mRNA) sequences. They have traditionally been used as research tools in biological experiments. For example, a morpholino oligomer can be used to block the expression of a particular gene in a cell. In this way, the function of a particular protein may be determined.

More recently, morpholino oligomer analogs of DNA have been used as possible therapeutics. For example, a number of morpholino oligomers are currently being developed to help people with Duchenne muscular dystrophy. Once administered to a Duchenne muscular dystrophy patient, these morpholino oligomers are designed to help the patient produce a shorter form of the dystrophin protein.

Currently, morpholino oligomers are generally synthesized using DNA bases (each base is either A, T, C, or G), but the disclosed method can be applied to other bases including U and modified bases such as methyl-U.

As the use of morpholino oligomers as therapeutic agents continues to grow, so does the need for systems and methods to quickly and accurately sequence their oligonucleotides. Mass spectrometry is a well-known method for sequencing oligonucleotides. However, to date, conventional mass spectrometry methods have been unable to properly fragment morpholino oligomers for sequencing.

As a result, there is a growing and unresolved need for mass spectrometry systems and methods to sequence morpholino oligomers.

### LC-MS and LC-MS/MS Background

Mass spectrometry (MS) is an analytical technique for the detection and quantitation of chemical compounds based on the analysis of mass-to-charge ratios (m/z) of ions formed from those compounds. The combination of mass spectrometry (MS) and liquid chromatography (LC) is an important analytical tool for the identification and quantitation of compounds within a mixture. Generally, in liquid chromatography, a fluid sample under analysis is passed through a column filled with a chemically-treated solid adsorbent material (typically in the form of small solid particles, e.g., silica). Due to slightly different interactions of components of the mixture with the solid adsorbent material (typically referred to as the stationary phase), the different components can have different transit (elution) times through the packed column, resulting in separation of the various components.

Note that for singly charged species, the terms “mass” and “m/z” are used interchangeably herein. One of ordinary skill in the art understands that a mass can be found from an m/z by multiplying the m/z by the charge. Similarly, the m/z can be found from a mass by dividing the mass by the charge.

In LC-MS, the effluent exiting the LC column can be continuously subjected to MS analysis. The data from this analysis can be processed to generate an extracted ion chromatogram (XIC), which can depict detected ion intensity (a measure of the number of detected ions of one or more particular analytes) as a function of retention time.

In MS analysis, an MS or precursor ion scan is performed at each interval of the separation for a mass range that includes the precursor ion. An MS scan includes the selection of a precursor ion or precursor ion range and mass analysis of the precursor ion or precursor ion range.

In some cases, the LC effluent can be subjected to tandem mass spectrometry (or mass spectrometry/mass spectrometry MS/MS) for the identification of product ions corresponding to the peaks in the XIC. For example, the precursor ions can be selected based on their mass/charge ratio to be subjected to subsequent stages of mass analysis. For example, the selected precursor ions can be fragmented (e.g., via collision-induced dissociation), and the fragmented ions (product ions) can be analyzed via a subsequent stage of mass spectrometry.

### Fragmentation Techniques Background

Electron-based dissociation (ExD), ultraviolet photodissociation (UVPD), infrared photodissociation (IRMPD), and collision-induced dissociation (CID) are often used as fragmentation techniques for tandem mass spectrometry (MS/MS). CID is the most conventional technique for dissociation in tandem mass spectrometers.

CID, in-source fragmentation, blackbody infrared radiative dissociation and IRMPD are examples of thermal-dissociation methods in this description. Thermal-dissociation methods included herein are non-radical dissociation methods that do not involve the use of radical formation in the dissociation process.

ExD can include, but is not limited to, electron-induced dissociation (EID), electron impact excitation in organics (EIEIO), electron capture dissociation (ECD), or electron transfer dissociation (ETD). Radical-induced dissociation methods, mentioned herein, include ExD, UVPD, electron detachment dissociation (EDD), plasma electron detachment dissociation (pEDD), and electron photo detachment dissociation (EPD).

### Tandem Mass Spectrometry or MS/MS Background

Tandem mass spectrometry or MS/MS involves ionization of one or more compounds of interest from a sample, selection of one or more precursor ions of the one or more compounds, fragmentation of the one or more precursor ions into product ions, and mass analysis of the product ions.

Tandem mass spectrometry can provide both qualitative and quantitative information. The product ion spectrum can be used to identify a molecule of interest. The intensity of one or more product ions can be used to quantitate the amount of the compound present in a sample.

A large number of different types of experimental methods or workflows can be performed using a tandem mass spectrometer. These workflows can include, but are not limited to, targeted acquisition, information dependent acquisition (IDA) or data dependent acquisition (DDA), and data independent acquisition (DIA).

In a targeted acquisition method, one or more transitions of a precursor ion to a product ion are predefined for a compound of interest. As a sample is being introduced into the tandem mass spectrometer, the one or more transitions are interrogated during each time period or cycle of a plurality of time periods or cycles. In other words, the mass spectrometer selects and fragments the precursor ion of each transition and performs a targeted mass analysis for the product ion of the transition. As a result, a chromatogram (the variation of the intensity with retention time) is produced for each transition. Targeted acquisition methods include, but are not limited to, multiple reaction monitoring (MRM) and selected reaction monitoring (SRM).

MRM experiments are typically performed using “low resolution” instruments that include, but are not limited to, triple quadrupole (QqQ) or quadrupole linear ion trap (QqLIT) devices. With the advent of “high resolution” instruments, there was a desire to collect MS and MS/MS using workflows that are similar to QqQ/QqLIT systems. High-resolution instruments include, but are not limited to, quadrupole time-of-flight (QqTOF) or orbitrap devices. These high-resolution instruments also provide new functionality.

MRM on QqQ/QqLIT systems is the standard mass spectrometric technique of choice for targeted quantification in all application areas, due to its ability to provide the highest specificity and sensitivity for the detection of specific components in complex mixtures. However, the speed and sensitivity of today's accurate mass systems have enabled a new quantification strategy with similar performance characteristics. In this strategy (termed MRM high resolution (MRM-HR) or parallel reaction monitoring (PRM)), looped MS/MS spectra are collected at high-resolution with short accumulation times, and then fragment ions (product ions) are extracted post-acquisition to generate MRM-like peaks for integration and quantification. With instrumentation like the TRIPLETOF® Systems of AB SCIEX™, this targeted technique is sensitive and fast enough to enable quantitative performance similar to higher-end triple quadrupole instruments, with full fragmentation data measured at high resolution and high mass accuracy.

In other words, in methods such as MRM-HR, a high-resolution precursor ion mass spectrum is obtained, one or more precursor ions are selected and fragmented, and a high-resolution full product ion spectrum is obtained for each selected precursor ion. A full product ion spectrum is collected for each selected precursor ion but a product ion mass of interest can be specified and everything other than the mass window of the product ion mass of interest can be discarded.

In an IDA (or DDA) method, a user can specify criteria for collecting mass spectra of product ions while a sample is being introduced into the tandem mass spectrometer. For example, in an IDA method a precursor ion or mass spectrometry (MS) survey scan is performed to generate a precursor ion peak list. The user can select criteria to filter the peak list for a subset of the precursor ions on the peak list. The survey scan and peak list are periodically refreshed or updated, and MS/MS is then performed on each precursor ion of the subset of precursor ions. A product ion spectrum is produced for each precursor ion. MS/MS is repeatedly performed on the precursor ions of the subset of precursor ions as the sample is being introduced into the tandem mass spectrometer.

In proteomics and many other applications, however, the complexity and dynamic range of compounds is very large. This poses challenges for traditional targeted and IDA methods, requiring very high-speed MS/MS acquisition to deeply interrogate the sample in order to both identify and quantify a broad range of analytes.

As a result, DIA methods, the third broad category of tandem mass spectrometry, were developed. These DIA methods have been used to increase the reproducibility and comprehensiveness of data collection from complex samples. DIA methods can also be called non-specific fragmentation methods. In a DIA method the actions of the tandem mass spectrometer are not varied among MS/MS scans based on data acquired in a previous precursor or survey scan. Instead, a precursor ion mass range is selected. A precursor ion mass selection window is then stepped across the precursor ion mass range. All precursor ions in the precursor ion mass selection window are fragmented and all of the product ions of all of the precursor ions in the precursor ion mass selection window are mass analyzed.

The precursor ion mass selection window used to scan the mass range can be narrow so that the likelihood of multiple precursors within the window is small. This type of DIA method is called, for example, MS/MS^{ALL}. In an MS/MS^{ALL} method, a precursor ion mass selection window of about 1 Da is scanned or stepped across an entire mass range. A product ion spectrum is produced for each 1 Da precursor mass window. The time it takes to analyze or scan the entire mass range once is referred to as one scan cycle. Scanning a narrow precursor ion mass selection window across a wide precursor ion mass range during each cycle, however, can take a long time and is not practical for some instruments and experiments.

As a result, a larger precursor ion mass selection window, or selection window with a greater width, is stepped across the entire precursor mass range. This type of DIA method is called, for example, SWATH acquisition. In a SWATH acquisition, the precursor ion mass selection window stepped across the precursor mass range in each cycle may have a width of 5-25 Da, or even larger. Like the MS/MS^{ALL} method, all of the precursor ions in each precursor ion mass selection window are fragmented, and all of the product ions of all of the precursor ions in each mass selection window are mass analyzed. However, because a wider precursor ion mass selection window is used, the cycle time can be significantly reduced in comparison to the cycle time of the MS/MS^{ALL} method.

U.S. Pat. No. 8,809,770 describes how SWATH acquisition can be used to provide quantitative and qualitative information about the precursor ions of compounds of interest. In particular, the product ions found from fragmenting a precursor ion mass selection window are compared to a database of known product ions of compounds of interest. In addition, ion traces or extracted ion chromatograms (XICs) of the product ions found from fragmenting a precursor ion mass selection window are analyzed to provide quantitative and qualitative information.

However, identifying compounds of interest in a sample analyzed using SWATH acquisition, for example, can be difficult. It can be difficult because either there is no precursor ion information provided with a precursor ion mass selection window to help determine the precursor ion that produces each product ion, or the precursor ion information provided is from a mass spectrometry (MS) observation that has a low sensitivity. In addition, because there is little or no specific precursor ion information provided with a precursor ion mass selection window, it is also difficult to determine if a product ion is convolved with or includes contributions from multiple precursor ions within the precursor ion mass selection window.

As a result, a method of scanning the precursor ion mass selection windows in SWATH acquisition, called scanning SWATH, was developed. Essentially, in scanning SWATH, a precursor ion mass selection window is scanned across a mass range so that successive windows have large areas of overlap and small areas of non-overlap. This scanning makes the resulting product ions a function of the scanned precursor ion mass selection windows. This additional information, in turn, can be used to identify the one or more precursor ions responsible for each product ion.

Scanning SWATH has been described in International Publication No. WO 2013/171459 A2 (hereinafter “the '459 Application”). In the '459 Application, a precursor ion mass selection window or precursor ion mass selection window of 25 Da is scanned with time such that the range of the precursor ion mass selection window changes with time. The timing at which product ions are detected is then correlated to the timing of the precursor ion mass selection window in which their precursor ions were transmitted.

The correlation is done by first plotting the mass-to-charge ratio (m/z) of each product ion detected as a function of the precursor ion m/z values transmitted by the quadrupole mass filter. Since the precursor ion mass selection window is scanned over time, the precursor ion m/z values transmitted by the quadrupole mass filter can also be thought of as times. The start and end times at which a particular product ion is detected are correlated to the start and end times at which its precursor is transmitted from the quadrupole. As a result, the start and end times of the product ion signals are used to determine the start and end times of their corresponding precursor ions.

### SUMMARY

In one aspect, the present disclosure generally relates to systems, methods and computer products for sequencing morpholino oligomers in which a mass spectrum of product ions generated via electron-based dissociation of a morpholino oligomer is determined and the mass spectrum is analyzed to determine a sequence of the morpholino oligomer.

In various embodiments, a mass spectrometric method for sequencing a morpholino oligomer is disclosed, which includes ionizing the morpholino oligomer to generate a positively-charged precursor ion, dissociating the positively-charged precursor ion using electron-based dissociation to generate a plurality of product ions, and determining one or more nucleotides of the morpholino oligomer based on analysis of m/z ratios of one or more of the product ions. By way of example, a mass spectrum of the product ions can be generated for determining their m/z ratios.

At least a portion of the product ions include backbone fragments of the morpholino precursor ion, i.e., the product ions are generated via breakage of a bond along the backbone of the morpholino oligomer.

In some embodiments, the electron-based dissociation, e.g., electron capture dissociation (ECD), is performed using electrons having an electron kinetic energy in a range of about 10 eV to about 20 eV. In other embodiments, the electron-based dissociation, e.g., ECD, is performed using electrons having an electron kinetic energy in a range of about 3 eV to about 7 eV. As discussed further below, the use of an electron kinetic energy in the higher range of about 10 eV to about 20 eV can lead to a more efficient dissociation (fragmentation) of a morpholino oligomer, thereby generating more fragment ions that can be analyzed for sequence determination and hence increasing the measurement sensitivity. However, the higher number of fragment ions can also increase the complexity of the resultant mass spectrum and render the requisite analysis more difficult and time consuming. The use of the lower kinetic energy range of about 3 eV to about 7 eV can, in turn, lead to a lower number of fragment ions and hence a lower complexity of the mass spectrum. In some embodiments, both of the above high and low kinetic energy ranges may be utilized in a structural analysis of a morpholino oligomer to generate complementary sets of data. In some embodiments, the information provided by the low and the high energy data sets can be combined to arrive at the sequence of the morpholino oligomer.

In various embodiments, the electron-based dissociation includes any of electron capture dissociation (ECD) and electron transfer dissociation with collisional post-activation (EThcD), and wherein optionally the electron capture dissociation comprises hot electron capture dissociation (hot ECD).

In some embodiments, the dissociation of the morpholino oligomer can be achieved via electrospray ionization.

In some embodiments, the m/z ratios of the d and z product ions and/or those of the b and x product ions can be utilized to determine one or more nucleotides of the morpholino oligomer, and more particularly a sequence of such nucleotides.

In some cases, the m/z ratios of the d and z product ions can be utilized as a primary information source for obtaining the structure of a morpholino oligomer and the m/z ratios of the b and x product ions can be employed as complementary information.

In some embodiments, at least a portion of a first set of product ions generated via dissociation of a plurality of morpholino oligomer precursor ions are caused to undergo another dissociation to generate a second set of product ions. By way of example, the dissociation of the first set of the product ions can be achieved via collision-induced dissociation (CID). By way of another example, the dissociation of the first set of product ions can be achieved using infrared multiple photo dissociation (IRMPD), e.g., by employing a CO₂ laser. Further, in some embodiments, subjecting a set of positively-charged precursor ions to an electron-based reaction (e.g., ETD) can lead to generation of charged reduced species corresponding to the precursor ions and/or product ions. In various embodiments, such charged-reduced species can be subjected to CID to generate a secondary set of product ions that can be detected and mass analyzed to facilitate the elucidation of the sequence of the morpholino oligomer.

In a related aspect, a mass spectrometric method for sequencing a morpholino oligomer is disclosed, which includes ionizing a plurality of morpholino oligomers to generate a plurality of positively-charged precursor ions, dissociating at least a first one of said positively-charged precursor ions using electron capture dissociation at an electron kinetic energy in a range of about 10 eV to about 20 eV to generate a first set of product ions, dissociating at least a second one of said positively-charged precursor ions using electron capture dissociation at an electron kinetic energy in a range of about 3 eV to about 7 eV to generate a second set of product ions, and determining a nucleotide sequence of the morpholino oligomer based on analysis of m/z ratios of one or more of the first set of the product ions and one or more of the second set of the product ions. By way of example, the mass spectra of the first and the second set of product ions can be generated to determine the m/z ratios of the product ions.

In a related aspect, a mass spectrometric method for sequencing a morpholino oligomer is disclosed, which includes using a processor to compute at least one mass spectrum of product ions generated via electron-based dissociation of a protonated morpholino oligomer and determining one or more nucleotides of the morpholino oligomer based on said at least one mass spectrum of the product ions. While in some embodiments, the electron-based dissociation (e.g., electron capture dissociation) is performed using electrons having a kinetic energy in a range of about 10 eV to about 20 eV, in other embodiments, a kinetic energy in a range of about 3 eV to about 7 eV can be utilized.

In some embodiments of the above method, at least one mass spectrum of the product ions can include at least two mass spectra, where one of the mass spectra is associated with product ions generated at an electron kinetic energy in a range of about 10 eV to about 20 eV and another one of the mass spectra is associated with product ions generated at an electron kinetic energy in a range of about 3 eV to about 7 eV.

In another related aspect, a computer system for sequencing a morpholino oligomer is disclosed, which includes a processor configured to receive ion detection data corresponding to a plurality of product ions generated via electron-based dissociation of a protonated morpholino oligomer and to compute at least one mass spectrum of the product ions, where the processor is configured to determine one or more nucleotides of the morpholino oligomer based on said at least one spectrum of the product ions. The processor can be further configured to determine a sequence of the one or more nucleotides of the morpholino oligomer.

By way of example, the electron-based dissociation can include electron capture dissociation (ECD) or electron transfer dissociation (ETD) with collisional post activation. Further, the electron-based dissociation can be performed using an electron kinetic energy in a range of about 10 eV to about 20 eV or in a range of about 3 eV to about 7 eV.

In some embodiments, the ion detection data received by the computer system can correspond to two sets of product ions, wherein one set of the product ions are generated at a kinetic energy in a range of about 10 eV to about 20 eV and another set of the product ions are generated at a kinetic energy in a range of about 3 eV to about 7 eV.

In yet another aspect, a mass spectrometric method for sequencing a morpholino oligomer is disclosed, which includes ionizing a plurality of morpholino oligomers in a sample to generate a plurality of positively-charged precursor ions corresponding to the morpholino oligomers and having different charge states (e.g., a charge state in a range from +7 to +12), obtaining a mass spectrum of the positively-charged precursor ions, identifying a mass peak with a maximum peak height in the mass spectrum of the positively-charged precursor ions, filtering the positively-charged precursor ions to select the precursor ion associated with the maximum mass peak, dissociating the selected precursor ion using electron-based dissociation, and determining one or more nucleotides of the morpholino oligomer based on analysis of m/z ratios of one or more of the product ions. In some embodiments, the step of determining the one or more nucleotides of the morpholino oligomer includes determining a sequence of the nucleotides. In some implementations of such a method, the electron-based dissociation can be any of electron capture dissociation (ECD) and electron transfer dissociation with collisional post activation (EThcD).

These and other features of the applicant's teachings are set forth herein.

### BRIEF DESCRIPTION OF THE DRAWINGS

The skilled artisan will understand that the drawings and appendices, described below, are for illustration purposes only. The drawings and appendices are not intended to limit the scope of the present teachings in any way.

FIG. 1A schematically depicts examples of modifications that can be made to RNA or DNA molecules to generate a variety of ONTs, including morpholino oligomers.

FIG. 1B is a block diagram that illustrates a computer system, upon which embodiments of the present teachings may be implemented. FIG. 1C is a flow chart depicting various steps in an embodiment of a method for sequencing a morpholino oligomer.

FIG. 2 is an exemplary product ion spectrum obtained from applying a resonant CID method to a morpholino oligomer analog of a DNA sequence.

FIG. 3 is an exemplary diagram showing the structure of a standard DNA molecule.

FIG. 4 is an exemplary diagram showing the structure of a morpholino oligomer analog of a DNA molecule.

FIG. 5 is an exemplary product ion spectrum obtained by applying an ECD method to fragment a morpholino oligomer analog of a DNA sequence, in accordance with various embodiments.

FIG. 6 is a schematic diagram of a system for sequencing a morpholino oligomer, in accordance with various embodiments.

FIG. 7 is an exemplary flowchart showing various steps of a method for sequencing a morpholino oligomer, in accordance with various embodiments.

FIG. 8 is a schematic diagram of a system that includes one or more distinct software modules and that performs a method for sequencing a morpholino oligomer, in accordance with various embodiments.

FIG. 9A is an example of a morpholino oligomer.

FIG. 9B shows the nomenclature of various backbone dissociations that can occur as the positively-charged morpholino oligomers are subjected to ECD.

FIG. 10A shows the chemical structure of a d fragment ion generated via dissociation of a morpholino oligomer.

FIG. 10B shows a mass spectrum of d fragment ions generated via backbone fragmentation of a morpholino oligomer at a fragmentation site #2.

FIG. 11A in turn shows the chemical structure of a ze fragment ion generated via dissociation of a morpholino oligomer.

FIG. 11B shows a mass spectrum of ze fragment ions generated via backbone fragmentation of a morpholino oligomer at a plurality of different fragmentation sites.

FIG. 12A shows the chemical structure of a b fragment ion generated via dissociation of a morpholino oligomer.

FIG. 12B shows a mass spectrum of b fragment ions generated via dissociation of a morpholino oligomer at a plurality of different fragmentation sites.

FIG. 13A shows the chemical structure of an x non-radical fragment ion generated via dissociation of a morpholino oligomer.

FIG. 13B shows a mass spectrum of x fragments ions generated via dissociation of a morpholino oligomer at a plurality of different fragmentation sites.

FIGS. 14A, 14B, 14C, and 14D show, respectively, fragment intensities of d, z⋅, b and x fragment ions that were generated via breakage of the bonds at different locations along the morpholino oligomer's backbone.

FIG. 15 shows a minor dissociation channel of a morpholino oligomer corresponding to NMe2 and base loss.

FIG. 16A presents a mass spectrum of product ions indicating that for a charge z=1, regular b fragment ions were observed.

FIG. 16B presents a mass spectrum of product ions indicating that for a charge state greater than 1, electron stripped radical fragment ions appear to be present.

FIGS. 17A and 17B present proposed structures of b and b⋅ ions.

FIGS. 18A, 18B, 18C, 18D, 18E, 18F and 18G show contamination of non-radical fragment species in radical z^{⋅} ions. The solid lines indicate m/z of the ¹³C profiles. The peaks at —H from the most left peak indicated solid line (i.e., mono isotopic peak of the radical z^{⋅} fragments) are the non-radical z ions, with FIG. 18A corresponding to z₁ (1+), FIG. 18B corresponding to z₃ (2+), FIG. 18C corresponding to z₂ (1+), FIG. 18D corresponding to z₄ (2+), FIG. 18E corresponding to z₅ (2+), FIG. 18F corresponding to z₆ (2+), and FIG. 18G corresponding to z₁₇ (5+).

FIGS. 19A and 19B illustrate, respectively, the structures of the ze and z ions.

FIGS. 20A and 20B show, respectively, the dependence of the production of z-H and ze ions on the kinetic energy of electrons (Ke) utilized in ECD dissociation of the precursor morpholino oligomers.

FIG. 21A shows raw intensities of the precursor and charge reduced species and FIG. 21B shows intensity-normalized Ke dependence.

FIGS. 22A, 22B, 22C, and 22D show representative mass spectra of product ions generated via ECD dissociation of a morpholino oligomer at the following kinetic energies of the electrons utilized in ECD: 5, 10, 15, 20 eV.

FIGS. 23A, 23B, 23C, and 23D show examples of mass spectra of product ions for low m/z ratios of the product ions at the following electron kinetic energies utilized in the ECD dissociation process: 5, 10, 15, and 20 eV.

FIGS. 24A, 24B, 24C, and 24D show examples of mass spectra of product ions for high m/z ratios of the product ions at the following electron kinetic energies utilized in the ECD dissociation process: 5, 10, 15, and 20 eV.

FIG. 25 shows that both low and high m/z regions, N-Me2(═NC2H6) loss and base loss were induced at high electron kinetic energies.

FIG. 26 shows an annotated product mass spectrum acquired at an electron kinetic energy of 15 eV.

FIG. 27 schematically depicts an ion trap and a downstream collision cell utilized in various embodiments for selecting certain ions subsequent to electron-based interactions for collisional dissociation.

FIG. 28 shows a mass spectrum of product ions generated via CID dissociation of a charge reduced species generated via charge reduction of a precursor ion.

FIGS. 29A, 29B, 29C, and 29D show intensity profiles, respectively, of d ions, ze ions, b ions, and x ions in ECD of a morpholino oligomer.

FIG. 30 illustrates examples of mass spectral data illustrating that CID does not produce backbone fragments from morpholino precursor ions.

FIG. 31 shows the mass spectrum of product ions generated via CID dissociation of a number of charge reduced species generated via subjecting morpholino precursor ions to electron-based interaction.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DETAILED DESCRIPTION

It will be appreciated that for clarity, the following discussion will explicate various aspects of embodiments of the applicant's teachings, while omitting certain specific details wherever convenient or appropriate to do so. For example, discussion of like or analogous features in alternative embodiments may be somewhat abbreviated. Well-known ideas or concepts may also for brevity not be discussed in any great detail. The skilled person will recognize that some embodiments of the applicant's teachings may not require certain of the specifically described details in every implementation, which are set forth herein only to provide a thorough understanding of the embodiments. Similarly, it will be apparent that the described embodiments may be susceptible to alteration or variation according to common general knowledge without departing from the scope of the dis closure. The following detailed description of embodiments is not to be regarded as limiting the scope of the applicant's teachings in any manner.

As used herein, the terms “about” and “substantially equal” refer to variations in a numerical quantity that can occur, for example, through measuring or handling procedures in the real world; through inadvertent error in these procedures; through differences in the manufacture, source, or purity of compositions or reagents; and the like. Typically, the terms “about” and “substantially” as used herein means 10% greater or less than the value or range of values stated or the complete condition or state. For instance, a concentration value of about 30% or substantially equal to 30% can mean a concentration between 27% and 33%. The terms also refer to variations that would be recognized by one skilled in the art as being equivalent so long as such variations do not encompass known values practiced by the prior art.

As used herein the term “and/or” includes any and all combinations of one or more of the associated listed items and may be abbreviated as “/”.

Oligonucleotide therapeutics (ONTs) are one of the new modalities that are being developed rapidly due to their high specificity and ability to reach previously untreatable targets. Most ONTs include modified nucleic acids to avoid degradation by nucleases, increase their stability, and improve specificity of a target sequence.

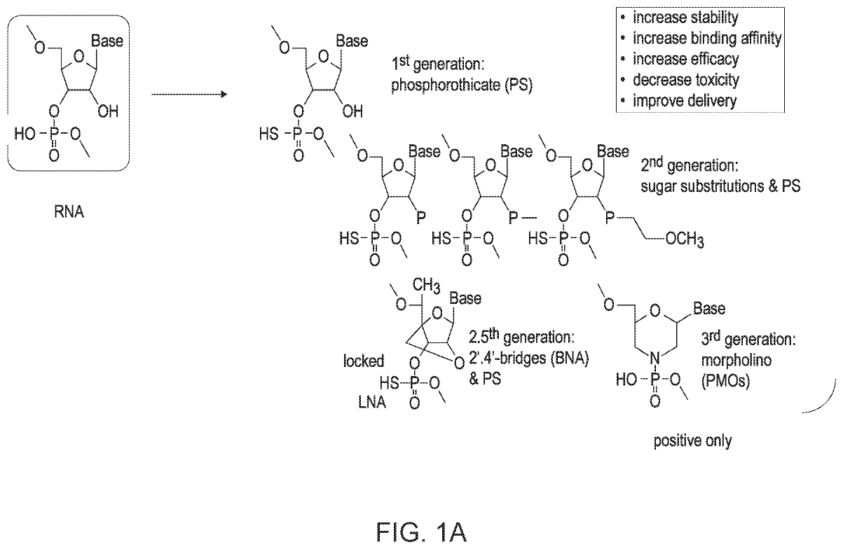

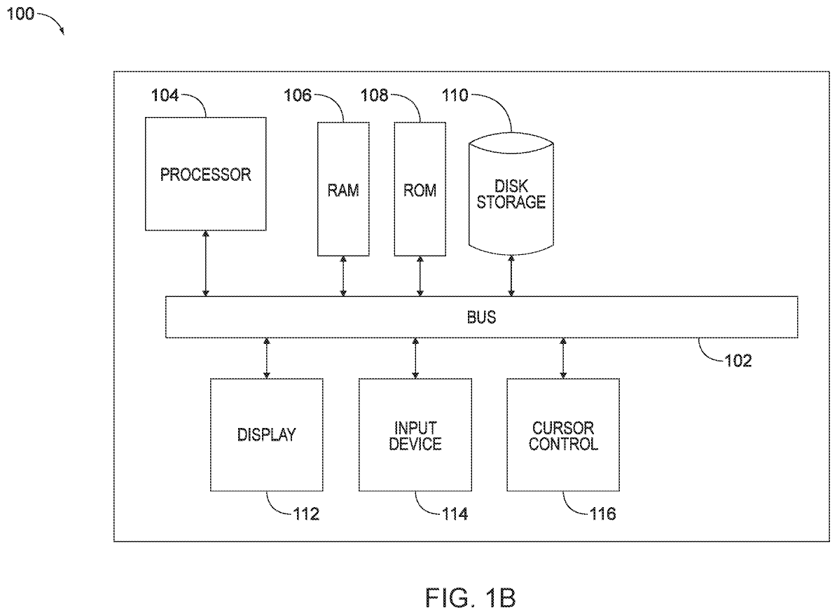

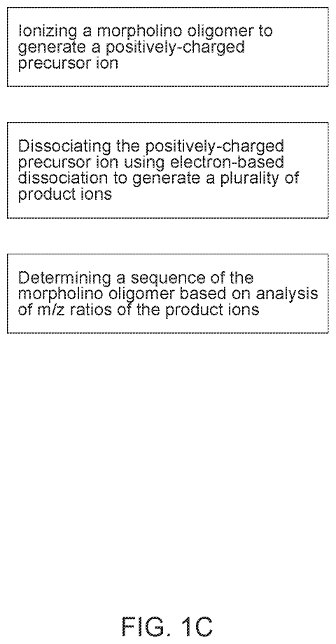

With reference to FIG. 1A, the first generation of ONTs included modifications at the phosphate groups of a DNA or RNA oligonucleotide. The second generation of ONTs included further modifications in the pentose groups. The third generation of ONTs include phosphorodiamidate morpholino oligomers (PMOs), which have a different structure with methylenemorpholine and phosphodiamidate backbone substitutions of conventional ribose-phosphate backbone. PMOs can work as ONTs and are nuclease-resistant.

ONTs are generally synthesized in the solid phase and can include some impurities. It is important to elucidate the structure of the ONTs and their impurities to ensure their safety and efficacy.

Although mass spectrometry has been used for acquiring structural information regarding conventional oligonucleotides, the use of mass spectrometry for elucidating the structure of morpholino oligomers poses certain challenges. For example, collision-induced dissociation does not cause backbone fragmentation of morpholino oligomers, and hence does not provide the requisite information for structural analysis.

Embodiments of systems and methods for sequencing a morpholino oligomer are provided herein. In this detailed description, for purposes of explanation, numerous specific details are set forth to provide a thorough understanding of embodiments of the present invention. One skilled in the art will appreciate, however, that embodiments of the present invention may be practiced without these specific details. In other instances, structures and devices are shown in block diagram form. Furthermore, one skilled in the art can readily appreciate that the specific sequences in which methods are presented and performed are illustrative and it is contemplated that the sequences can be varied and remain within the spirit and scope of embodiments of the present invention.

It has been discovered that mass spectrometric structural analysis of morpholino oligomers can be accomplished via ionization of the morpholino oligomers to generate protonated precursor ions, subjecting such protonated precursor ions to electron-based dissociation to generate a plurality of product ions, and analyzing the mass spectrum of the product ions. As discussed in more detail below, in some cases, electron-based activation (such as electron transfer dissociation) together with collisional post activation may be utilized for generating the product ions.

In various embodiments, one or both of two energy regimes for the kinetic energy of electrons employed for the electron-based dissociation can be utilized. In one such electron energy regime, the kinetic energy of the electrons can be in a range of about 10 eV to about 20 eV while in the other electron energy regime, the kinetic energy of the electrons can be in a range of about 3 eV to about 7 eV. The use of the higher electron energy range can result in a greater degree of dissociation of a protonated morpholino oligomer and hence an improved measurement sensitivity, albeit at a higher complexity of the mass spectrum. On the other hand, the use of the lower electron kinetic energy can lead to a lower complexity of the resultant mass spectrum, albeit at a lower degree of oligomer dissociation. In some embodiments, a sample under study can be analyzed using both energy regimes, e.g., a portion of the sample can be analyzed using electron energy in a range of about 10 eV to about 20 eV and another portion of the sample can be analyzed using electron energy in a range of about 3 eV to about 7 eV. The mass spectrometric information gleaned from the data acquired in these two energy regimes may be combined to obtain a more accurate determination of the structure of the morpholino oligomer.

### Computer-Implemented System

FIG. 1B is a block diagram that illustrates a computer system 100, upon which embodiments of the present teachings may be implemented. Computer system 100 includes a bus 102 or other communication mechanism for communicating information, and a processor 104 coupled with bus 102 for processing information. Computer system 100 also includes a memory 106, which can be a random-access memory (RAM) or other dynamic storage device, coupled to bus 102 for storing instructions to be executed by processor 104. Memory 106 also may be used for storing temporary variables or other intermediate information during execution of instructions to be executed by processor 104. Computer system 100 further includes a read only memory (ROM) 108 or other static storage device coupled to bus 102 for storing static information and instructions for processor 104. A storage device 110, such as a magnetic disk or optical disk, is provided and coupled to bus 102 for storing information and instructions.

Computer system 100 may be coupled via bus 102 to a display 112, such as a cathode ray tube (CRT) or liquid crystal display (LCD), for displaying information to a computer user. An input device 114, including alphanumeric and other keys, is coupled to bus 102 for communicating information and command selections to processor 104. Another type of user input device is cursor control 116, such as a mouse, a trackball or cursor direction keys for communicating direction information and command selections to processor 104 and for controlling cursor movement on display 112.

A computer system 100 can perform the present teachings. Consistent with certain implementations of the present teachings, results are provided by computer system 100 in response to processor 104 executing one or more sequences of one or more instructions contained in memory 106. Such instructions may be read into memory 106 from another computer-readable medium, such as storage device 110. Execution of the sequences of instructions contained in memory 106 causes processor 104 to perform the process described herein.

Alternatively, hard-wired circuitry may be used in place of or in combination with software instructions to implement the present teachings. For example, the present teachings may also be implemented with programmable artificial intelligence (AI) chips with only the encoder neural network programmed—to allow for performance and decreased cost. Thus, implementations of the present teachings are not limited to any specific combination of hardware circuitry and software.

The term “computer-readable medium” or “computer program product” as used herein refers to any media that participates in providing instructions to processor 104 for execution. The terms “computer-readable medium” and “computer program product” are used interchangeably throughout this written description. Such a medium may take many forms, including but not limited to, non-volatile media and volatile media. Non-volatile media includes, for example, optical or magnetic disks, such as storage device 110. Volatile media includes dynamic memory, such as memory 106.

Common forms of computer-readable media include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 104 for execution. For example, the instructions may initially be carried on the magnetic disk of a remote computer. The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector coupled to bus 102 can receive the data carried in the infra-red signal and place the data on bus 102. Bus 102 carries the data to memory 106, from which processor 104 retrieves and executes the instructions. The instructions received by memory 106 may optionally be stored on storage device 110 either before or after execution by processor 104.

In accordance with various embodiments, instructions configured to be executed by a processor to perform a method are stored on a computer-readable medium. The computer-readable medium can be a device that stores digital information. The computer-readable medium is accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software but the present teachings may be implemented as a combination of hardware and software or in hardware alone. The present teachings may be implemented with both object-oriented and non-object-oriented programming systems.

### Morpholino Electron Capture Dissociation

As described above, morpholino oligonucleotides are synthetic oligonucleotides that have traditionally been used as research tools in biological experiments.

More recently, however, morpholino oligomers are being explored as possible therapeutics.

As the use of morpholino oligomers as therapeutic agents continues to grow, so does the need for systems and methods to quickly and accurately sequence their oligonucleotides. Mass spectrometry is a well-known method for sequencing oligonucleotides. However, to date, conventional mass spectrometry methods have been unable to properly fragment morpholino oligomers for sequencing.

Thus, there is a growing and unresolved need for mass spectrometry systems and methods to sequence morpholino oligomers.

FIG. 1C is a flow chart depicting various steps of a method according to an embodiment for sequencing a morpholino oligomer. The method includes ionizing the morpholino oligomer to generate a positively-charged precursor ion and dissociating the positively-charged precursor ion using electron-based dissociation to generate a plurality of product ions. The morpholino oligomer's sequence can be determined based on analysis of m/z ratios of the one or more of the product ions.

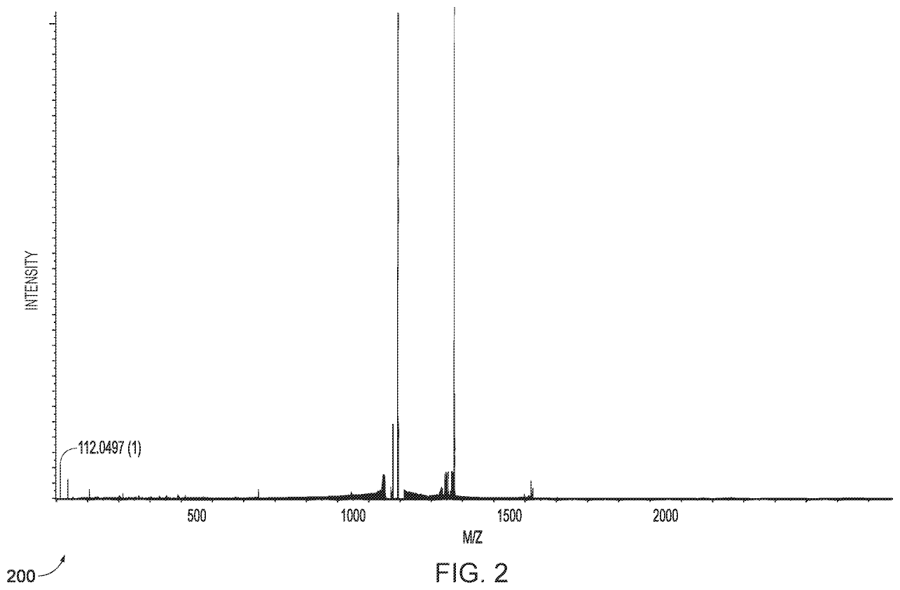

FIG. 2 is an exemplary product ion spectrum 200 obtained from applying a resonant CID method to fragment a morpholino oligomer. FIG. 2 shows no backbone fragmentation of the morpholino oligomer. As a result, CID cannot be used to obtain the information needed for sequencing morpholino oligomers.

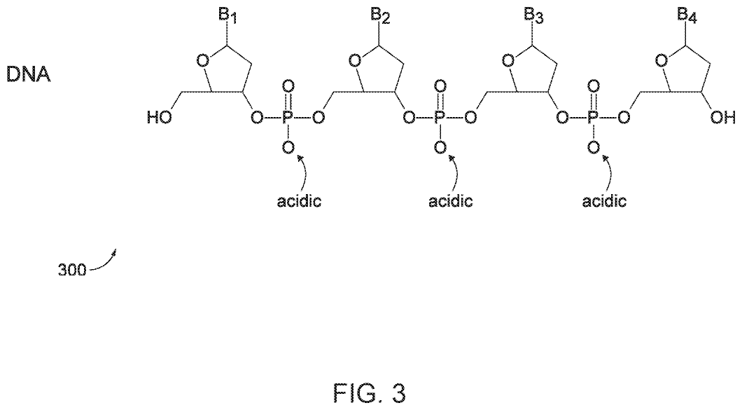

Moreover, electron detachment dissociation (EDD), which can be employed for determining structure of a DNA molecule, is not suitable for fragmenting morpholino oligomer precursor ions. By way of illustration, FIG. 3 provides an exemplary diagram 300 showing the structure of a DNA molecule, indicating that the nucleotides of the DNA molecule can be ionized to acquire a negative charge. The negatively charged DNA molecule can undergo dissociation via EDD or CID.

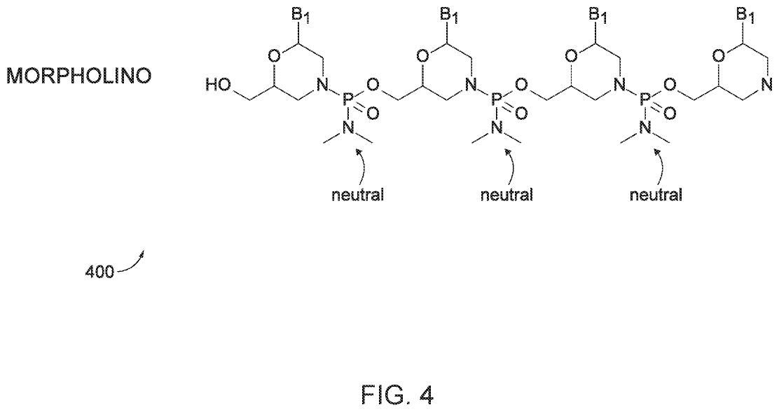

FIG. 4 is, in turn, an exemplary diagram 400 showing the structure of a morpholino oligomer analog of a DNA molecule. FIG. 4 shows that the nucleotides of morpholino oligomer are ionized positively, e.g., via electrospray ionization. As a result, mass spectrometry methods, such as EDD, that require that the precursor ions be negatively charged are unable to fragment morpholino oligomers.

In various embodiments, morpholino oligomers are fragmented and sequenced using ECD. In particular, it has been discovered that in various embodiments the application of ECD to positively charged morpholino analogs of DNA can produce a complete series of backbone fragments (herein also referred to as product ions), which can facilitate the sequencing of the morpholino oligomer.

In various embodiments, a morpholino oligomer can then be sequenced based on mass spectrometric information (e.g., the mass spectrum) corresponding to d, z and ze ions produced via ECD fragmentation of the positively-charged morpholino oligomers. While in some embodiments the sequencing of the morpholino oligomers can be performed in real-time during product ion spectrum acquisition, in other embodiments, post-acquisition processing of the ion detection data can be utilized to sequence the morpholino oligomers.

As noted above, in various embodiments, the electron beam energy of the ECD is 0-5 eV. In various alternative embodiments, the electron beam energy of the ECD is 10-20 eV.

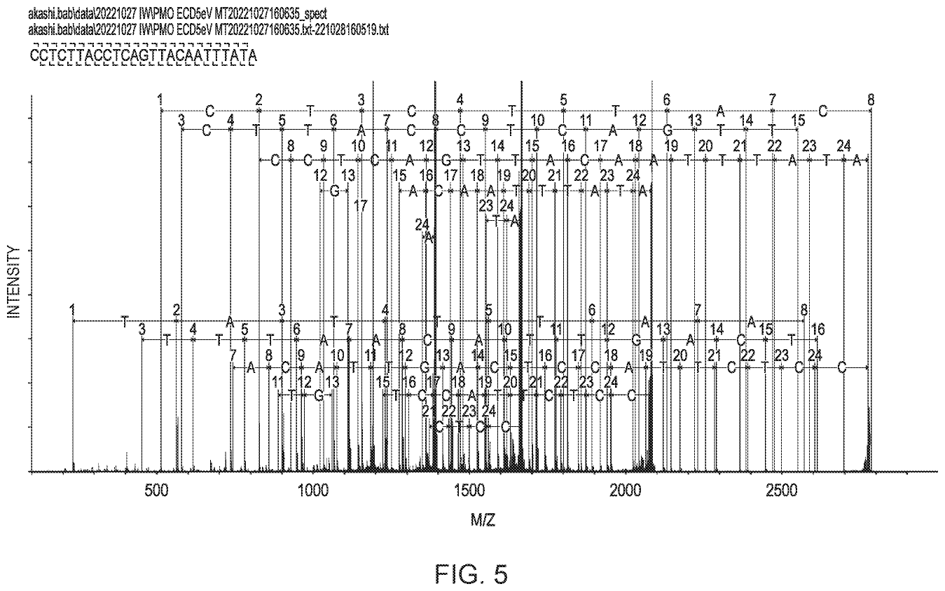

FIG. 5 presents an exemplary product ion spectrum 500 obtained by applying an ECD method to fragment a morpholino oligomer, in accordance with various embodiments. FIG. 5 shows that in this example, ECD dissociation of the positively-charged precursor ion produced a complete series of backbone product ions. From these d and ze ions, a sequence for the morpholino oligomer can be determined.

### System for Sequencing a Morpholino Oligomer

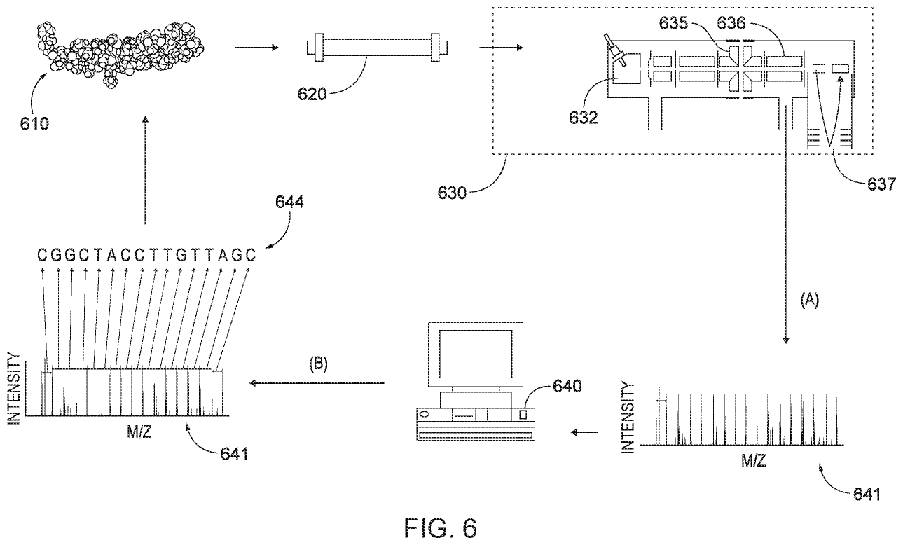

FIG. 6 is a schematic diagram 600 of a system for sequencing a morpholino oligomer, in accordance with various embodiments. The system includes processor 640, which can be, but is not limited to, a controller, a computer, a microprocessor, such as the computer system of FIG. 1B, or any device capable of analyzing data. Processor 640 can also be any device capable of sending and receiving control signals and data. In various embodiments, in step (A), processor 640 receives a mass spectrum 641 of a plurality of product ions of a morpholino oligomer 610, where the product ions were produced via electron-based dissociation of positively-charged precursor ions formed via ionization of the morpholino oligomers.

In step (B), processor 640 determines one or more nucleotides of the morpholino oligomer from product ion mass spectrum 641. Sequence 644 is produced for morpholino oligomer 610, for example.

In various embodiments, a precursor ion of morpholino oligomer 610 includes a protonated morpholino oligomer produced by electrospray ionization.

In various embodiments, a positively-charged morpholino precursor ion is dissociated to generate a plurality of product ions using electron-based dissociation, such as ECD, or electron transfer dissociation with collisional post activation (EThcCD). In various embodiments, the electron-based dissociation mass spectrometry can further include applying CID-based post-activation to ECD product ions to generate another set of product ions (herein also referred to as secondary product ions).

As noted above, in various embodiments, the electron kinetic energy of the ECD is 5 eV±2 eV. In various other embodiments, the electron kinetic energy of the ECD is 15 eV±5 eV.

In various embodiments, the electron-based dissociation mass spectrometry further includes applying CID-based post-activation to dissociate charged reduced product ions. Post activation can increase the intensities of d and ze ions, thereby increasing the sensitivity of sensitivity of the associated sequencing method.

In various embodiments, the activation energy of the CID is 20 eV±5 eV.

In various embodiments, the electron-based dissociation mass spectrometry further includes applying infrared multiple photon dissociation (IRMPD)-based post-activation to ECD product ions.

In various embodiments, the electron-based dissociation mass spectrometry further includes applying infrared multiple photon dissociation (IRMPD)-based post-activation to isolated charged reduced product ions.

In various embodiments, the IRMPD is produced using a CO₂ laser.

In various embodiments, the electron-based dissociation mass spectrometry includes hot electron capture dissociation (hot ECD).

In various embodiments, the electron-based dissociation mass spectrometry includes electron transfer dissociation with collisional post-activation (EThcD).

In various embodiments, in step (B), d and z⋅ product ions of product ion spectrum 641 are used to determine the one or more nucleotides of the morpholino oligomer.

In various embodiments, in step (B), b and x product ions of product ion spectrum 641 are used to determine the one or more nucleotides of the morpholino oligomer.

In various embodiments, the system of FIG. 6 further includes mass spectrometer 630. Ion source device 632 of mass spectrometer 630 ionizes the morpholino oligomer 610, producing an ion beam. Ion source device 632 is controlled by processor 640, for example. Ion source device 632 is shown as a component of mass spectrometer 630. In various alternative embodiments, ion source device 632 is a separate device. Ion source device 632 can be, but is not limited to, an electrospray ion source (ESI) device or a chemical ionization (CI) source device such as an atmospheric pressure chemical ionization source (APCI) device or an atmospheric pressure photoionization (APPI) source device.

Mass spectrometer 630 selects and fragments morpholino oligomer 610 and mass analyzes product ions of morpholino oligomer 610 from the ion beam. Mass spectrometer 630 further includes CID device 636, electron-based dissociation device 635, and mass analyzer 637. Mass spectrometer 630 produces mass spectrum 641 using electron-based dissociation device 635.

In FIG. 6, mass analyzer 637 is shown as a time-of-flight (TOF) device. One of ordinary skill in the art can appreciate that mass analyzer 637 can be any type of mass analyzer including, but not limited to, a quadrupole, an ion trap, an orbitrap, or a Fourier transform ion cyclotron resonance (FT-ICR) device. In various embodiments, the system of FIG. 6 further includes a separation device 620 that separates morpholino oligomer 610 from a sample. As shown in FIG. 6, the additional device 620 is an LC device. In various alternative embodiments, additional device 620 can be, but is not limited to, a gas chromatography (GC) device, capillary electrophoresis (CE) device, or an ion mobility spectrometry (IMS) device.

### Method for Sequencing a Morpholino Oligomer

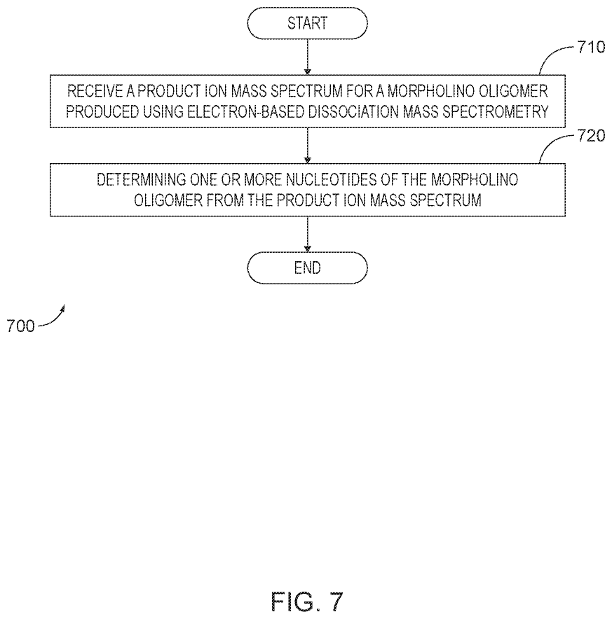

FIG. 7 is an exemplary flowchart showing a method 700 for sequencing a morpholino oligomer, in accordance with various embodiments.

In step 710 of method 700, a product ion mass spectrum for a morpholino oligomer produced using electron-based dissociation mass spectrometry is received.

In step 720, one or more nucleotides of the morpholino oligomer are determined from the product ion mass spectrum.

### Computer Program Product for Sequencing a Morpholino Oligomer

In various embodiments, a computer program product includes a non-transitory tangible computer-readable storage medium whose contents include a program with instructions being executed on a processor so as to perform a method for sequencing a morpholino oligomer. This method is performed by a system that includes one or more distinct software modules.

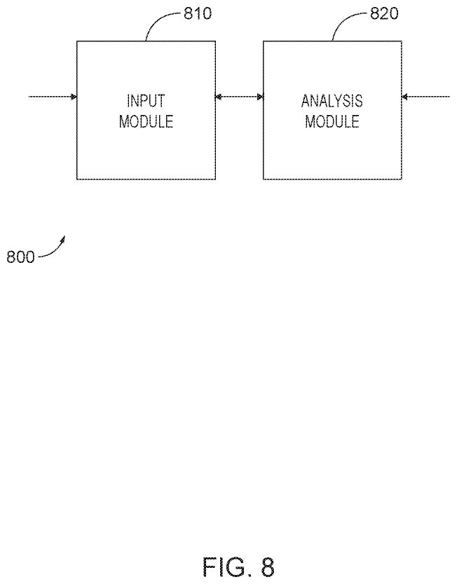

FIG. 8 is a schematic diagram of a system 800 that includes one or more distinct software modules and that performs a method for sequencing a morpholino oligomer, in accordance with various embodiments. System 800 includes input module 810 and analysis module 820.

In step (A), input module 810 receives a product ion mass spectrum for a morpholino oligomer produced using electron-based dissociation mass spectrometry.

In step (B), analysis module 820 determines one or more nucleotides of the morpholino oligomer from the product ion mass spectrum.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The following examples are provided for further elucidation of various aspects of the present teaching and are not intended to indicate necessarily optimal ways of practicing the present teachings and/or optimal results that may be obtained.

### EXAMPLES

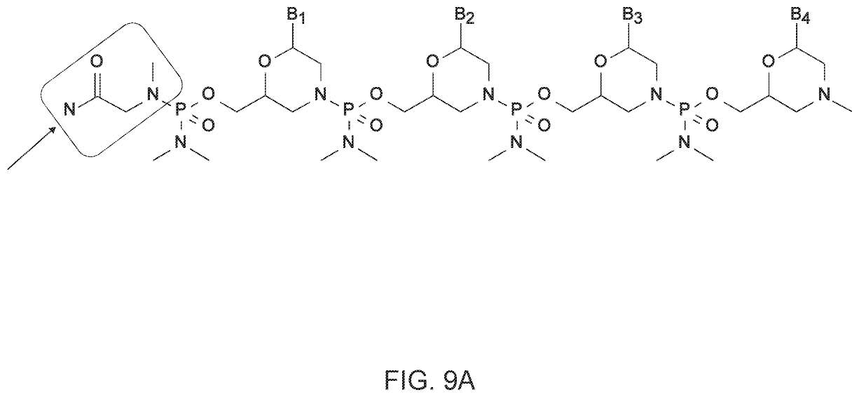

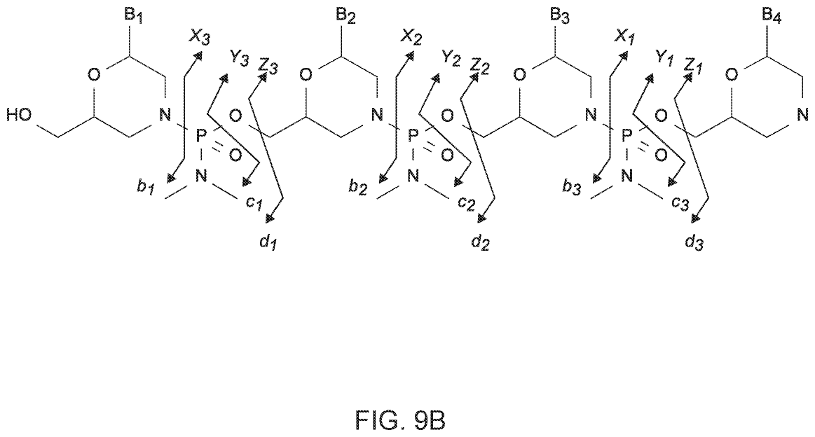

Morpholino standard was obtained from Gene Tools with the following nucleotide sequence was obtained: CCTCCTTACCTCAGTTACAATTTATA. As shown in FIG. 9A, the morpholino sample had a linker at its 5′ terminus.

A working solution of the morpholino at a concentration of 10 μg/mL in MeOH: distilled water: formic acid (50:50:0.1) was prepared. The working solution was infused into a Research grade Sciex mass spectrometer having an ECD cell and a time-of-flight (TOF) mass analyzer at an infusion rate of 5 μL/min. Electron spray ionization (ESI) was employed for the ionization of the morpholino oligomers. The ionization conditions are summarized in Table 1 below:

TABLE 1

| | | | Ion | on | | | Ionspray |
|---|---|---|---|---|---|---|---|
| Infusion | ESi | | source | source | Curtain | | Voltage |
| rate | voltage | DP | gas 1 | gas 2 | gas | Temperature | Floating |
| 5 μL/min | 5500 V | 120 V | 50 psi | 30 psi | 30 psi | 250 °C | 5500 V |

It was discovered that ECD can cause dissociation of the morpholino oligomer so as to produce backbone fragments. FIG. 9B shows the nomenclature of various backbone dissociations that can occur as the positively-charged morpholino oligomers are subjected to ECD.

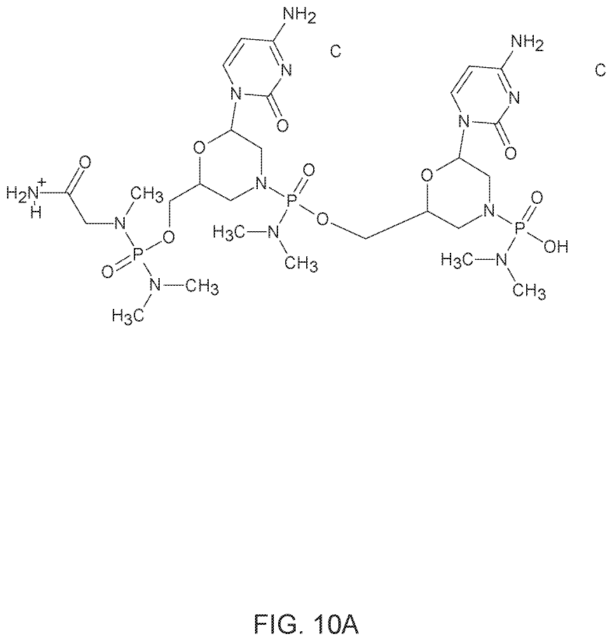

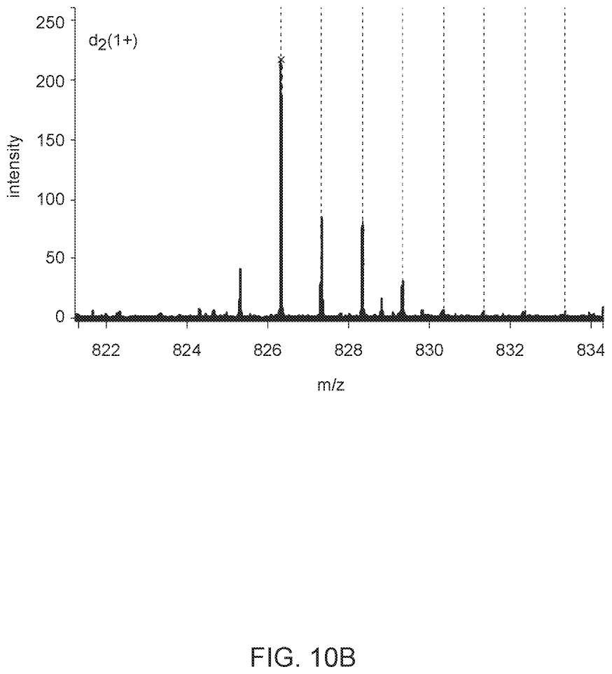

FIG. 10A shows a proposed chemical structure of a d fragment ion, which is a non-radical fragment with a theoretical m/z ratio of 826.3038, and FIG. 10B shows the mass spectrum of the d fragment ions generated via backbone bond cleavages at different sites. The proposed chemical structure needs to be experimentally and/or theoretically validated.

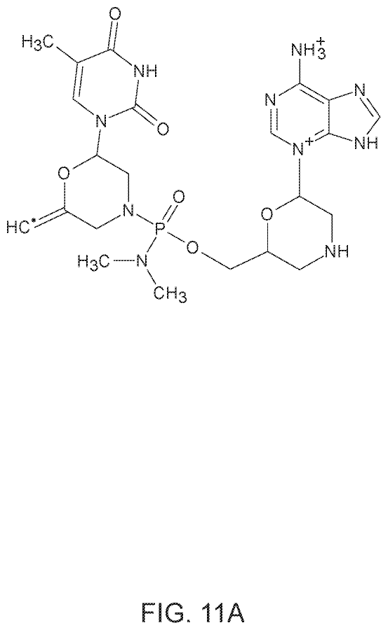

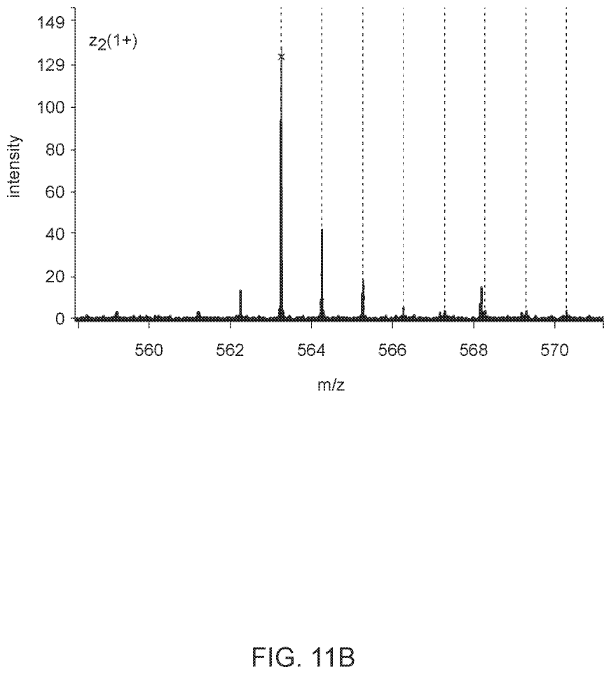

FIG. 11A in turn shows a proposed chemical structure of a z⋅ fragment, which is a radical, with a theoretical m/z ratio of 563.2233. And FIG. 11B shows the corresponding mass spectrum of the ze fragment ions generated via backbone bond cleavages at different sites.

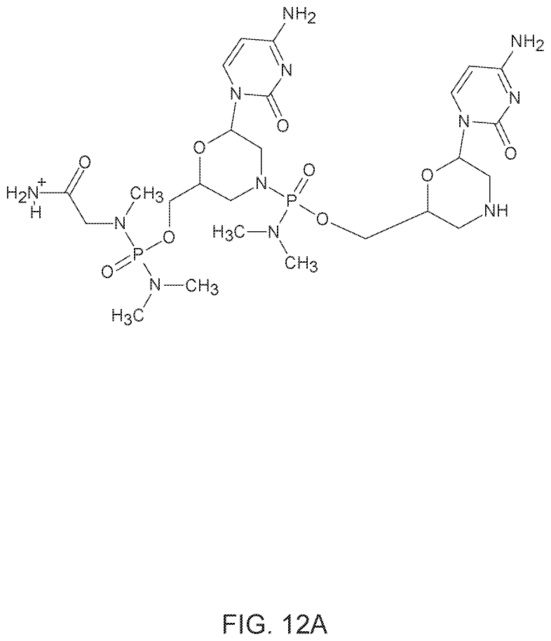

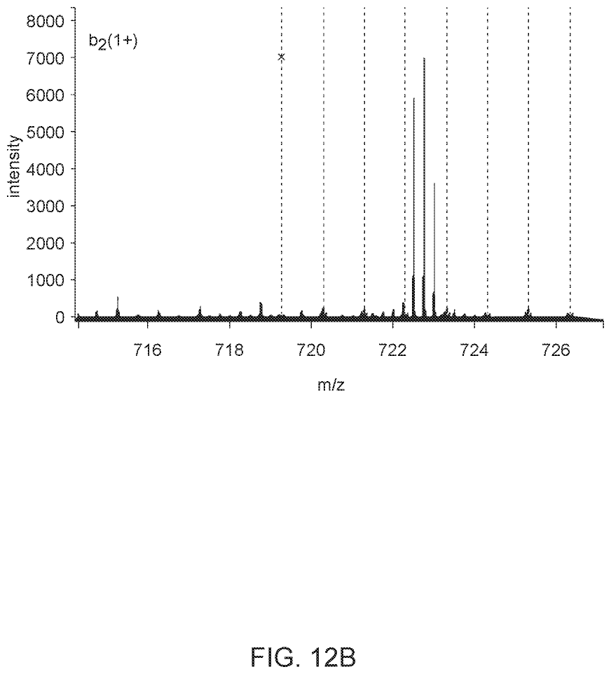

FIG. 12A shows the a proposed chemical structure of a b fragment, which is a non-radical with a theoretical m/z ratio of 719.29071. FIG. 12B shows the corresponding mass spectrum of the b fragment ions generated via backbone bond cleavages at different sites.

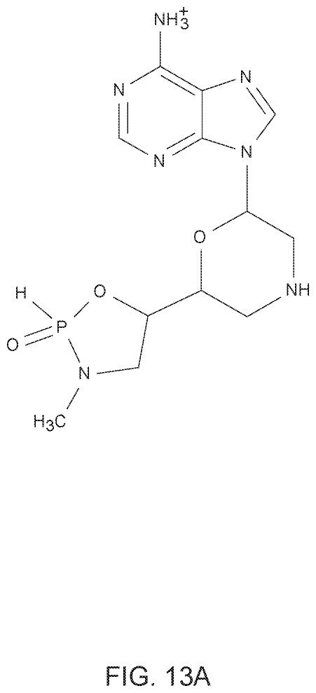

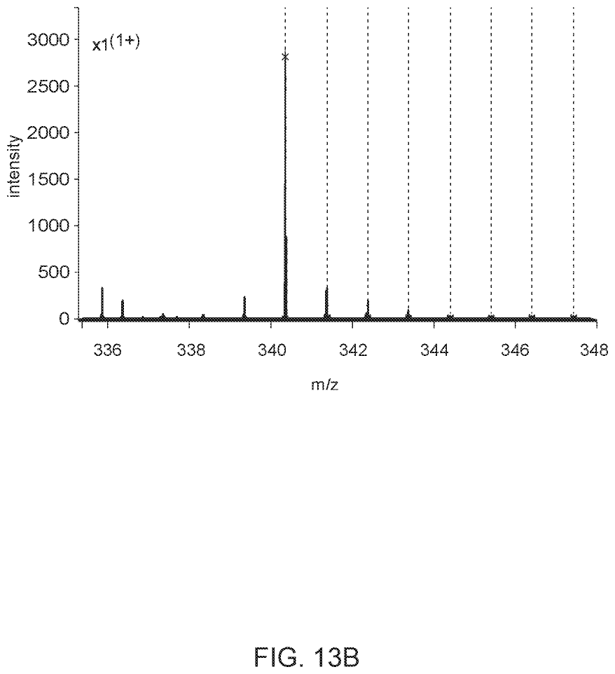

FIG. 13A shows a proposed chemical structure of an x non-radical fragment ion with a theoretical m/z ratio of 340.12869. FIG. 13B shows the mass spectrum of the x fragment ions generated via backbone bond cleavages at different sites.

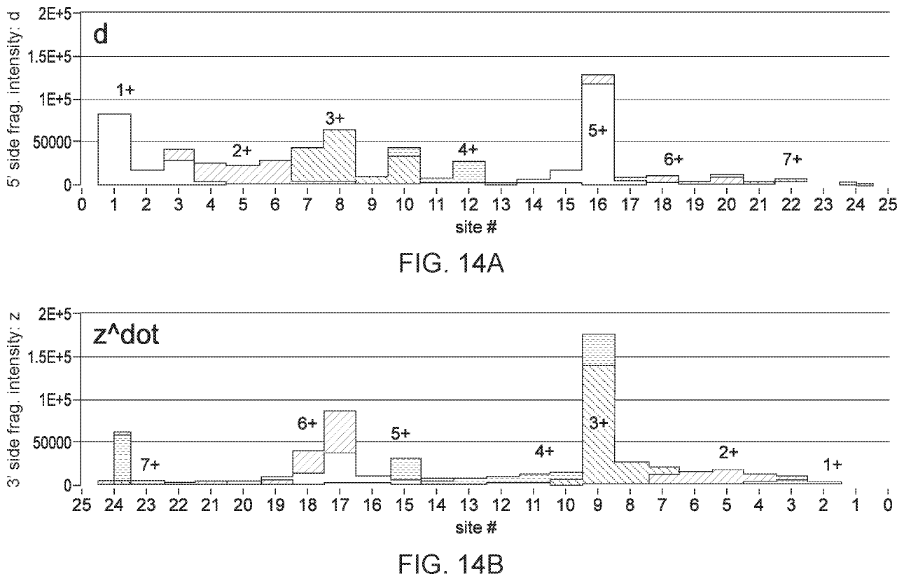

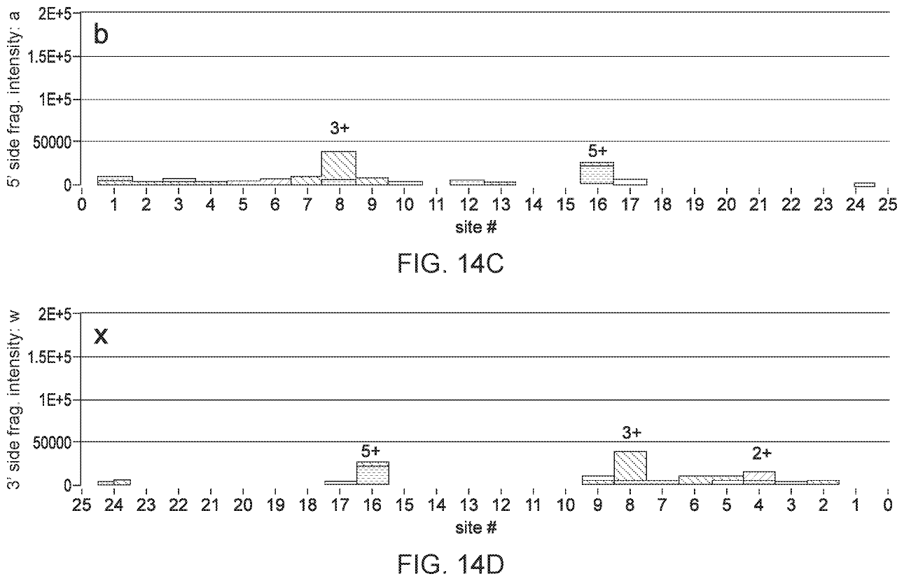

FIGS. 14A, 14B, 14C, and 14D show fragment intensities of d, z⋅, b and x fragment ions that were generated via breakage of the bonds at different locations along the morpholino oligomer's backbone. Charge states are indicated in the graphs of FIGS. 14A, 14B, 14C, and 14D.

The above data shows that the main fragmentation channel leads to the formation of d and z⋅ fragment ions. The b and x fragment ions are in turn generated via a sub fragmentation process. The intensities of the d and z⋅ are substantially uniform, but with d16 and z⋅9 fragment ions exhibiting high intensities.

The mass spectrometric information associated with the d and z⋅ fragment ions were used for data interpretation and in particular for sequencing of the morpholino oligomer. Further, the mass spectrometric information associated with the b and x fragment ions provided complementary information and were used to supplement the mass spectrometric data associated with the d and z⋅ fragment ions.

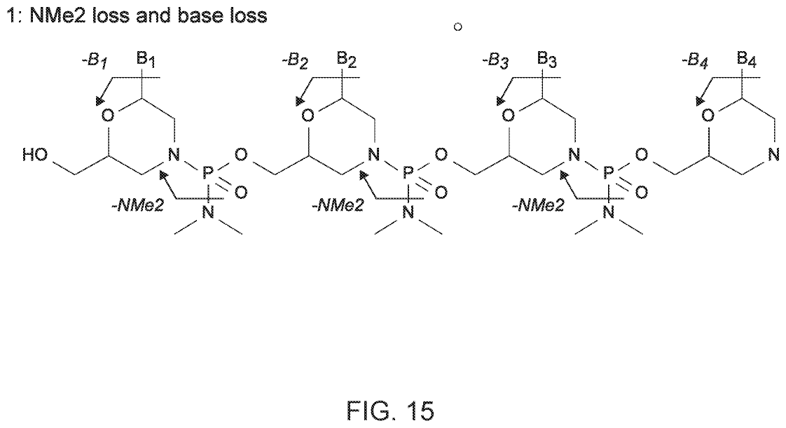

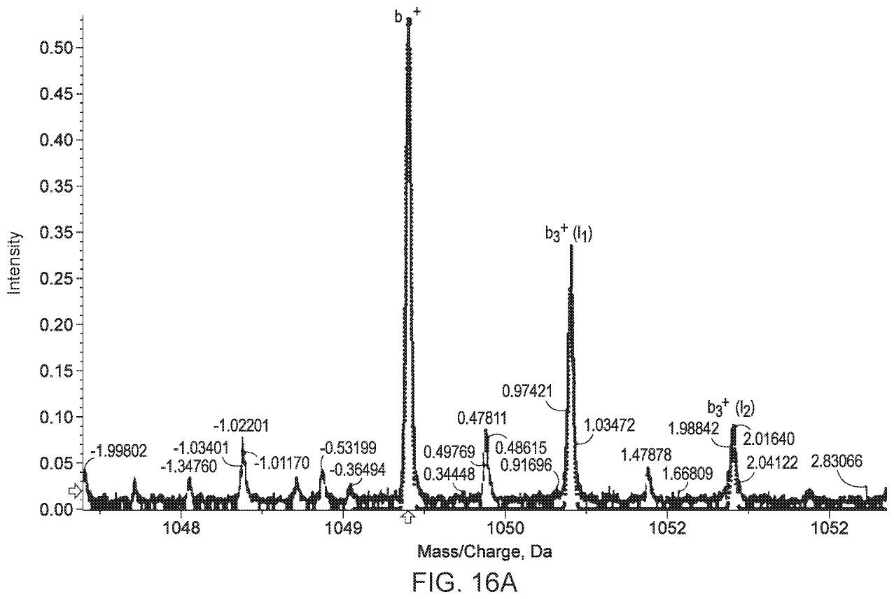

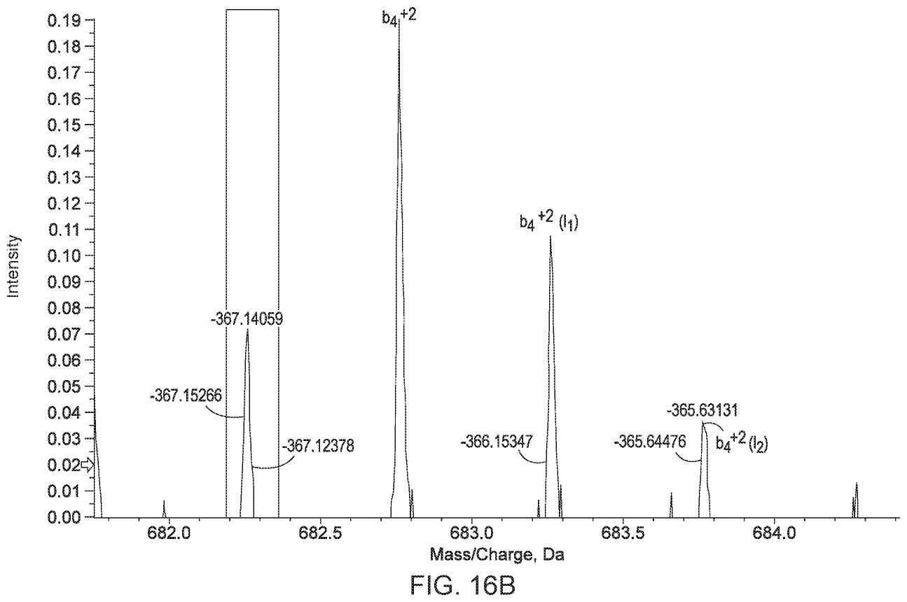

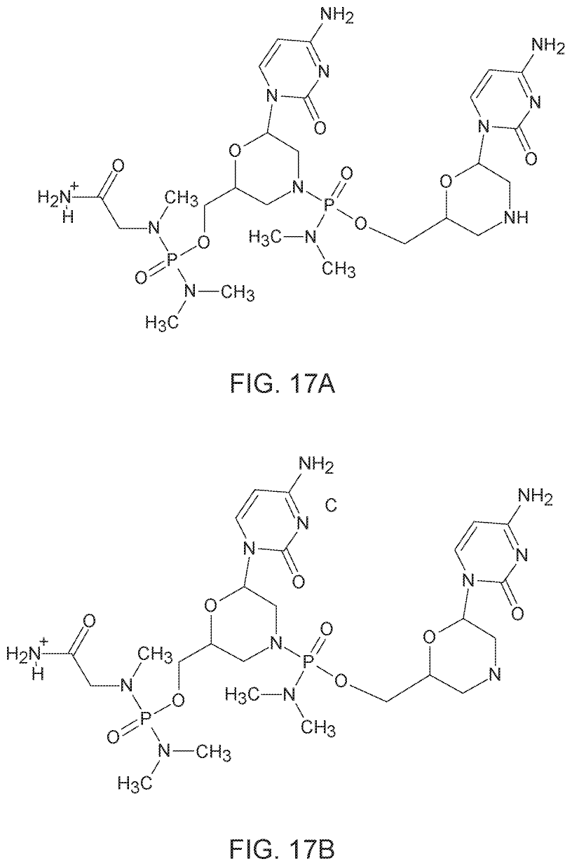

The following minor dissociation channels were also identified. In particular, FIG. 15 shows a minor dissociation channel corresponding to NMe2 and base loss. Moreover, radical-to-non-radical transitions were observed. For example, with reference to FIGS. 16A and 16B, for a charge state z=1, regular b fragments were observed whereas for a change state greater than 1 (i.e., z>1), electron stripped radical fragments appear to be present. Proposed structures of b and b⋅ ions are illustrated in FIGS. 17A and 17B.

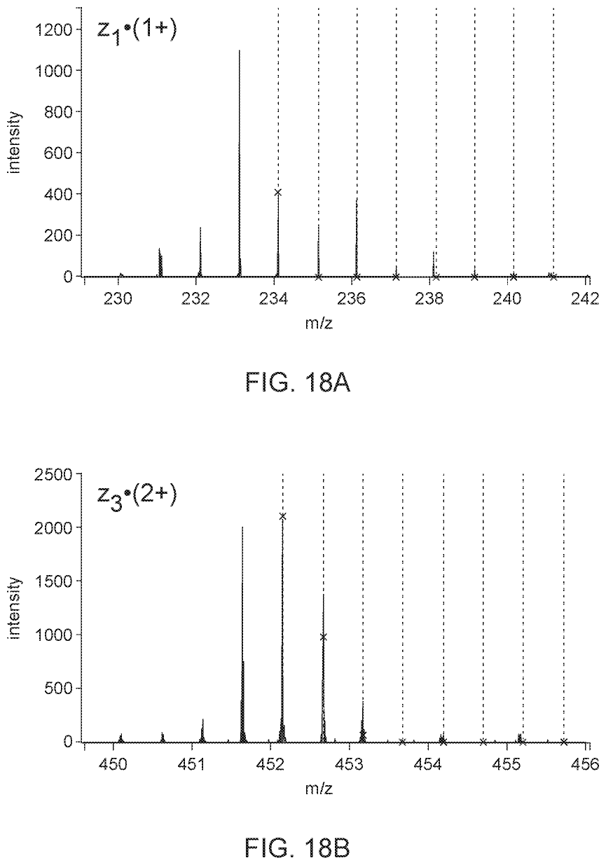

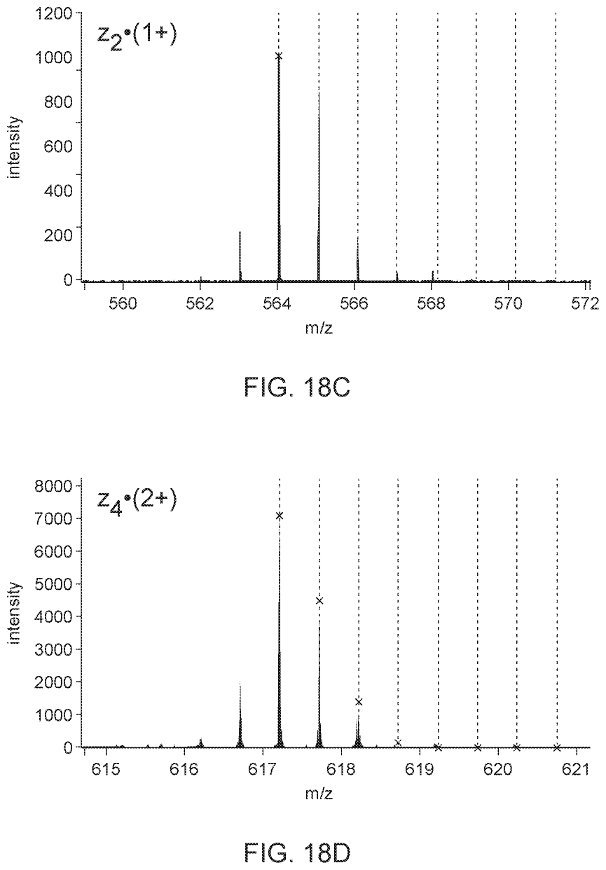

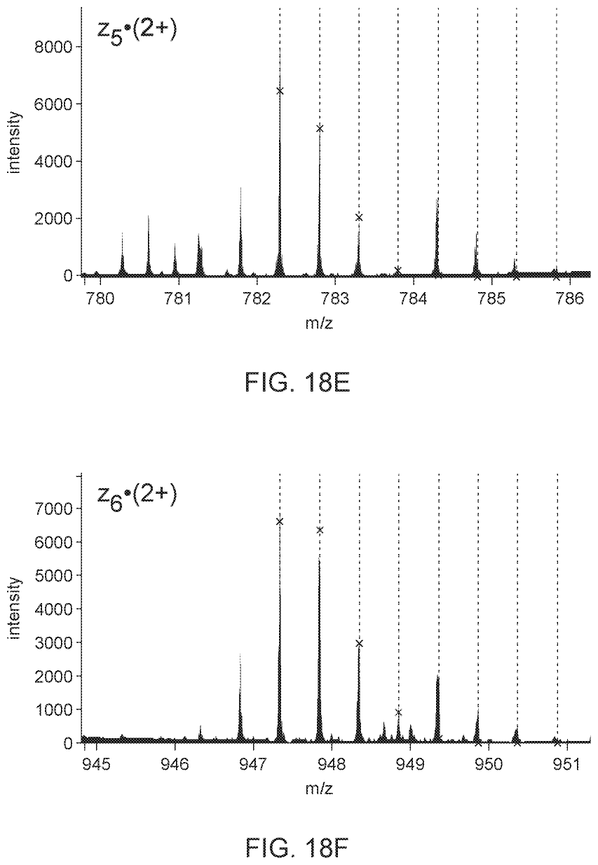

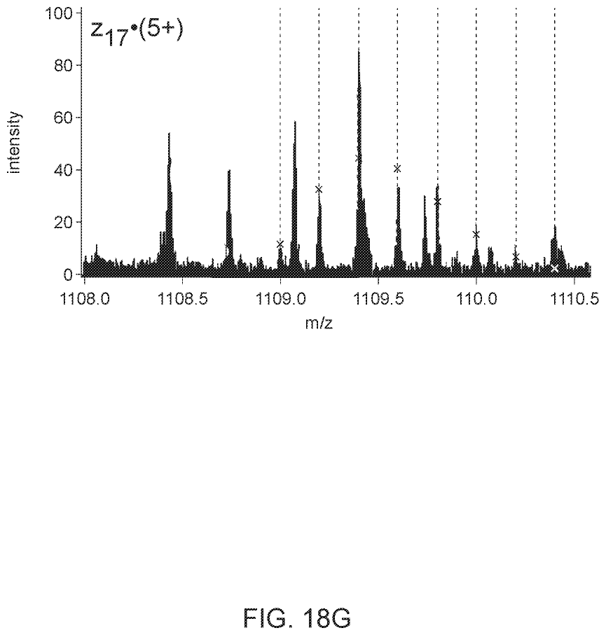

The data shows that high charge density z⋅-H may be produced. In the case a z⋅ fragment has a low charge density, the lifetime of the associated intermediate species is expected to be too long to allow migration of a hydrogen. FIGS. 18A, 18B, 18C, 18D, 18E, 18F and 18G show contamination of non-radical fragment species in radical z^{⋅} ions. The solid lines indicate m/z of the ¹³C profiles. The peaks at —H from the most left peak indicated the solid line (i.e., mono isotopic peak of the radical z^{⋅} fragments) are the non-radical z ions. 18A: z₁ (1+), 18B: z₃ (2+), 18C: z₂ (1+), 18D: z₄ (2+), 18E: z₅ (2+), 18F: z₆ (2+), and 18G: z₁₇ (5+). In a low charge density case (18G), the ¹³C profile is matched to pure radical z ions without —H products. When the charge density is increasing (18C-18F), the intensities of the hydrogen lost species are increasing. In the highest charge densities (18A and 18B), the hydrogen lost species are more intense than the radical z′ ions.

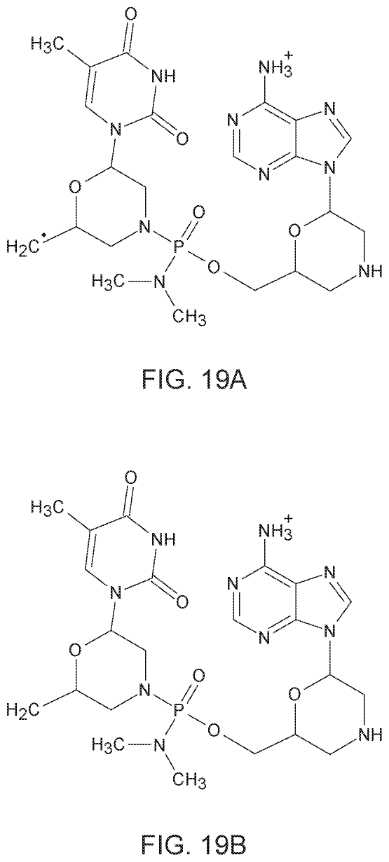

FIGS. 19A and 19B illustrate, respectively, the structures of the ze and z ions.

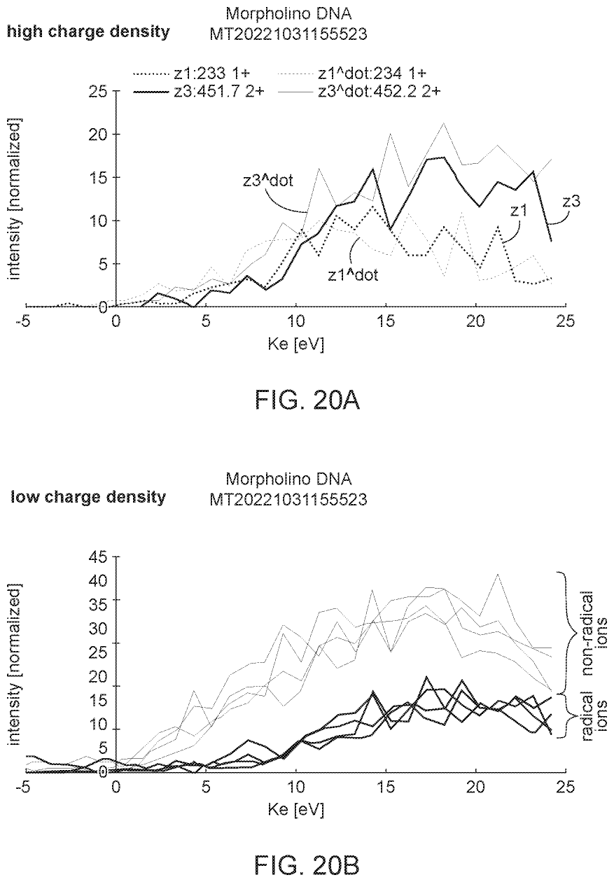

FIGS. 20A and 20B show the dependence of the production of z-H and z⋅ ions on the kinetic energy of electrons (Ke) utilized in ECD dissociation of the precursor morpholino oligomers. The data shows that the production of high charge density fragment ions require an electron kinetic energy greater than about 5 eV, with the z-H product ions being co-produced with radical z ions. Intensities of z-H and z⋅ ions were similar.

At lower charge density, ze ions were produced with electron kinetic energy greater than 0 eV and z-H ions were produced in Ke>7 eV. The production of the z-H ions was less than that of the z⋅ ions.

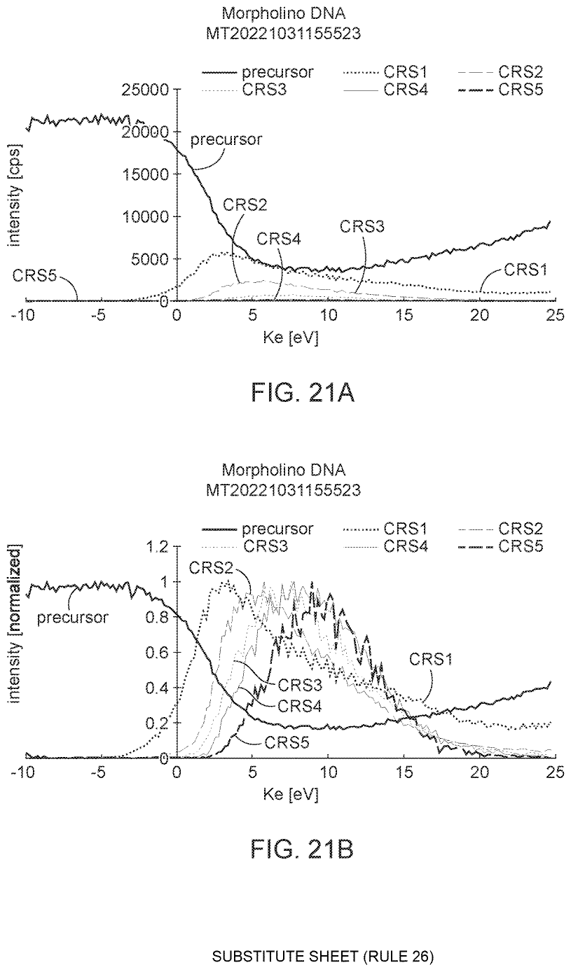

FIG. 21A shows raw intensities of the precursor and charge reduced species. FIG. 21B shows intensity-normalized Ke dependence. The data did not show electron capture at 0 eV, which appears in ECD of peptides and proteins. However, hot ECD in the energy range of 3 eV to 15 eV was confirmed.

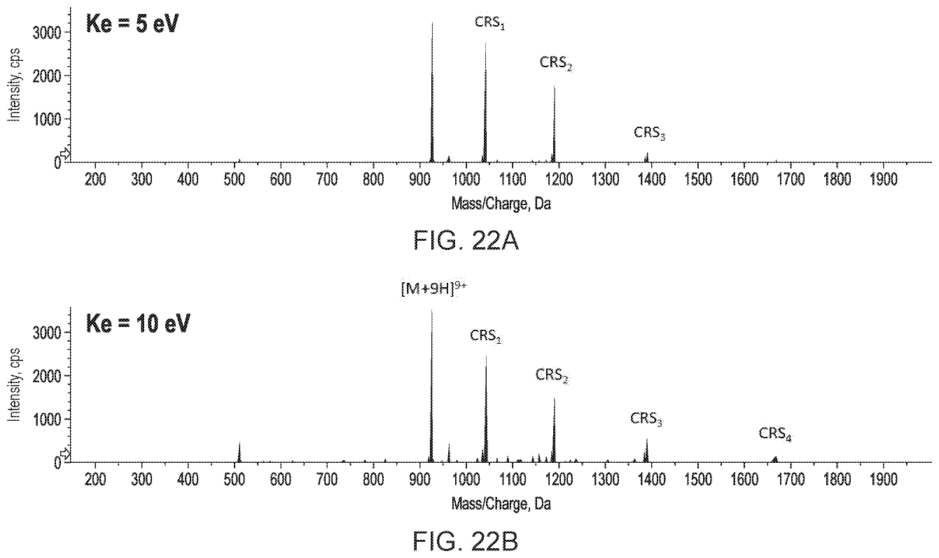

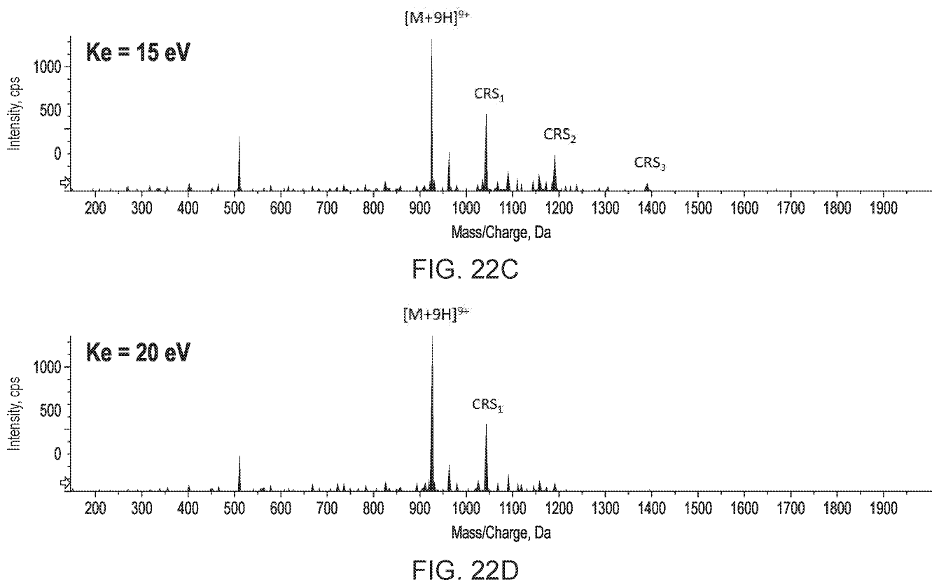

FIGS. 22A, 22B, 22C, and 22D show representative mass spectra of product ions generated via ECD dissociation of a morpholino oligomer at the following kinetic energies of the electrons utilized in ECD: 5, 10, 15, 20 eV. Further, an electron kinetic energy of 15±2 eV was used to obtain hot ECD data.

The data shows that multiple charge reduced species (CRS) as well as undissociated morpholino precursor ions can be suppressed at higher electron kinetic energies. In particular, this can be achieved by utilizing electron kinetic energies in a range of about 10 eV to about 20 eV, with the kinetic energy at 15 eV providing the best results. The kinetic energy of 15±2 eV was used for obtaining hot ECD data.

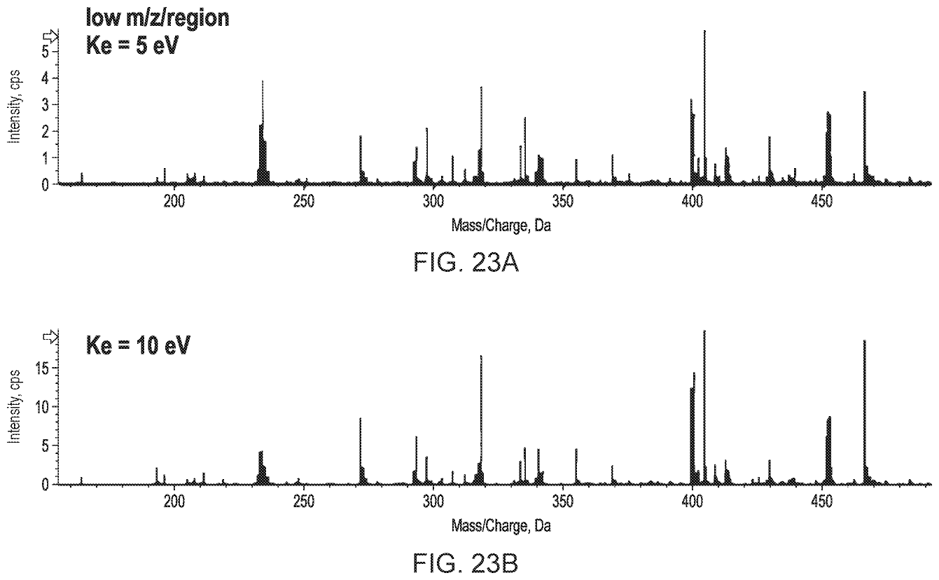

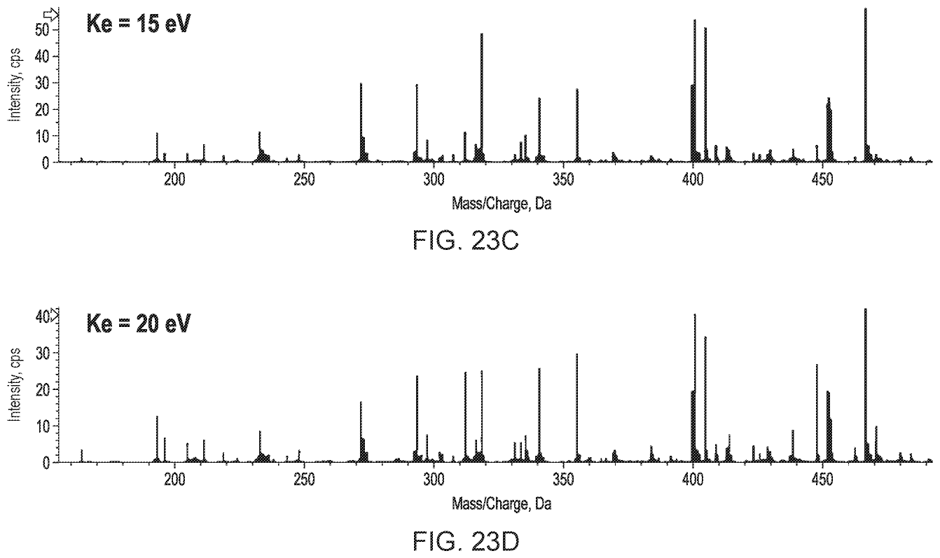

FIGS. 23A, 23B, 23C, and 23D show examples of mass spectra of product ions for low m/z ratios of the product ions at the following electron kinetic energies utilized in the ECD dissociation process: 5, 10, 15, and 20 eV. The mass data in the low m/z region exhibit more internal/lost fragments at higher electron kinetic energies.

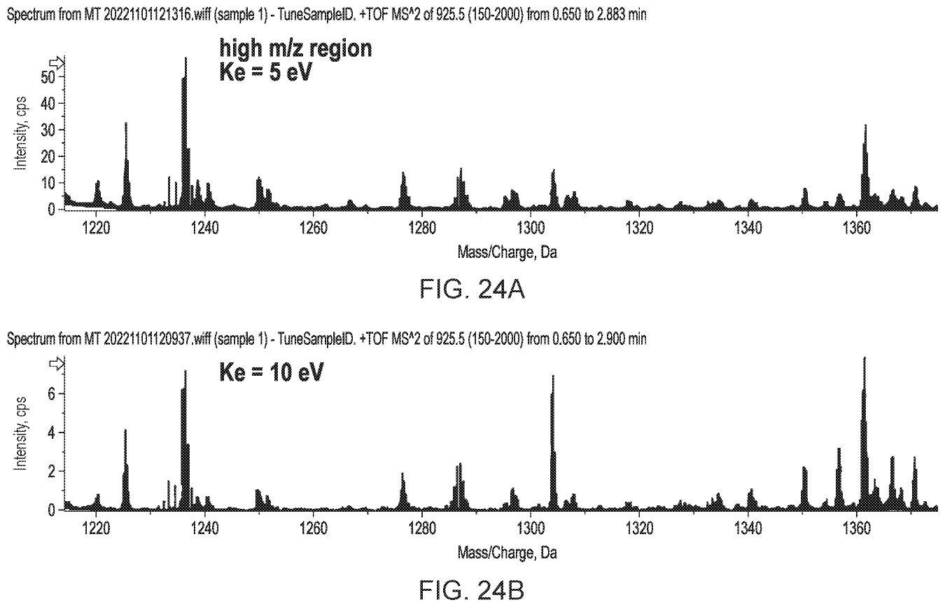

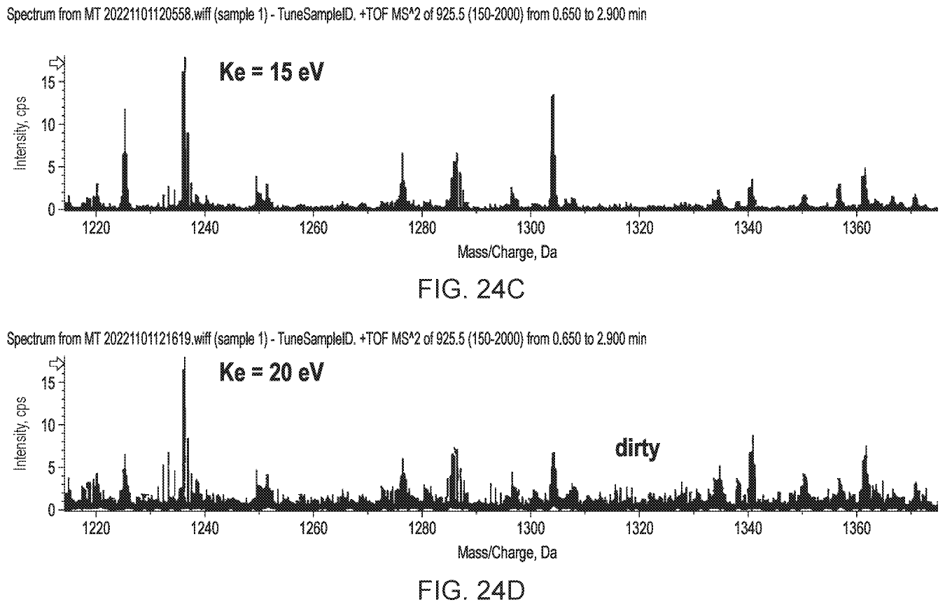

FIGS. 24A, 24B, 24C, and 24D show examples of mass spectra of product ions for high m/z ratios of the product ions at the following electron kinetic energies utilized in the ECD dissociation process: 5, 10, 15, and 20 eV. A strong 1 Da ladder background (periodic spikes in the spectrum) is observed at the electron kinetic energy of 20 eV so KE of 20 eV should be too high. In this example, optimal results were obtained at an energy of 15 eV because at this energy the peak intensities are high but the 1 Da ladder background will be negligible in data analysis.

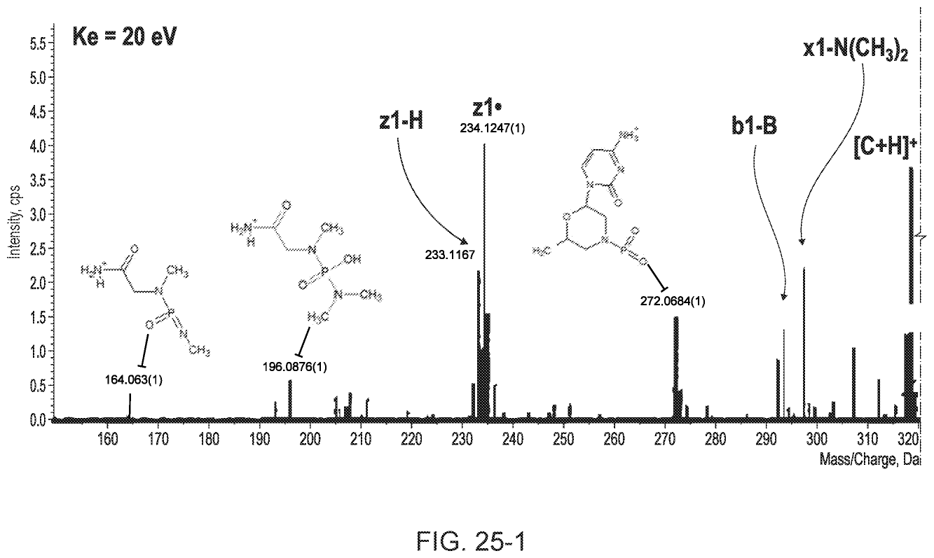

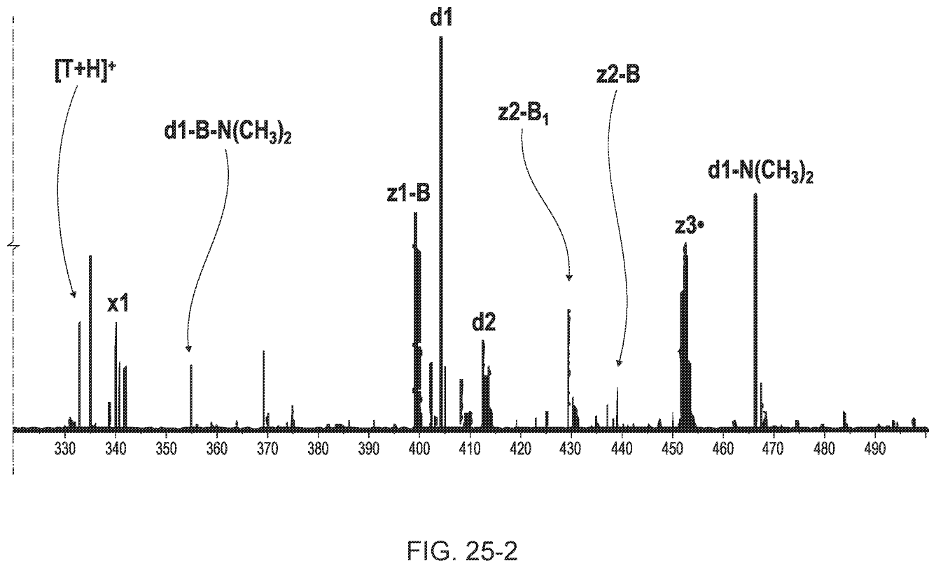

With reference to FIG. 25, it is noted that in both low and high m/z regions, N-Me2(═NC2H6) loss and base loss were induced at high electron kinetic energies.

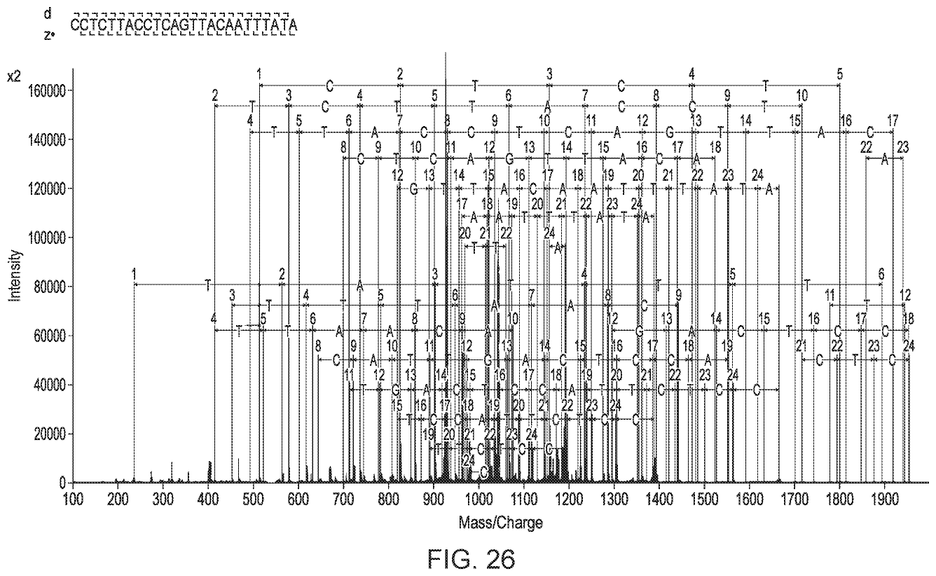

FIG. 26 shows the annotated product mass spectrum acquired at an electron kinetic energy of 15 eV. The spectrum shows a sequence coverage of 100%, thus indicating that an electron kinetic energy of 15 eV can be particularly suitable for determining the sequence of a morpholino oligomer.

The above data shows that almost no fragment ions were generated at Ke=0 eV. The above data also shows that in these examples, the 1 Da ladder can be avoided by using an electron kinetic energy of 5 eV. More generally, as discussed above, it has been discovered that less congested mass spectra, which allow a more facile analysis of the mass data, can be acquired by using electron kinetic energies in a range of 5±2 eV.

### Post-Activation on CRS1

### Example 1

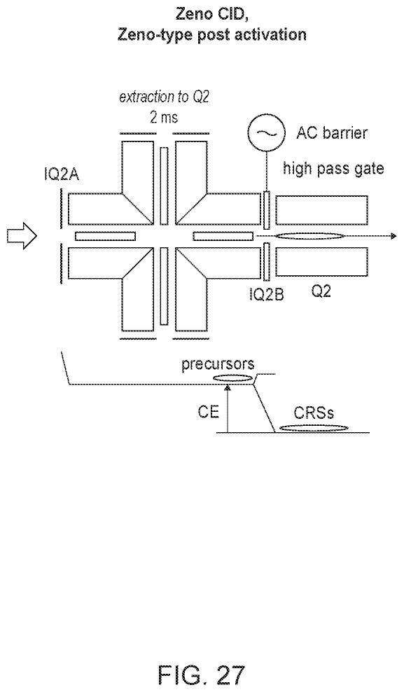

As discussed above, in some cases, some precursor ions can undergo charge reduction to generate charge reduced species (CRS) via electron capture. Such charge reduced species can be isolated and dissociated using collision induced dissociation (CID). For example, the charged reduced species can be isolated using a quadrupole mass filter and then introduced into an ion trap. With reference to FIG. 27, selected ions, such as charge-reduced species, can be extracted from the ion trap and introduced into a downstream collision cell to cause fragmentation of those ions (or at least a portion thereof). such as the ion trap illustrated in FIG.

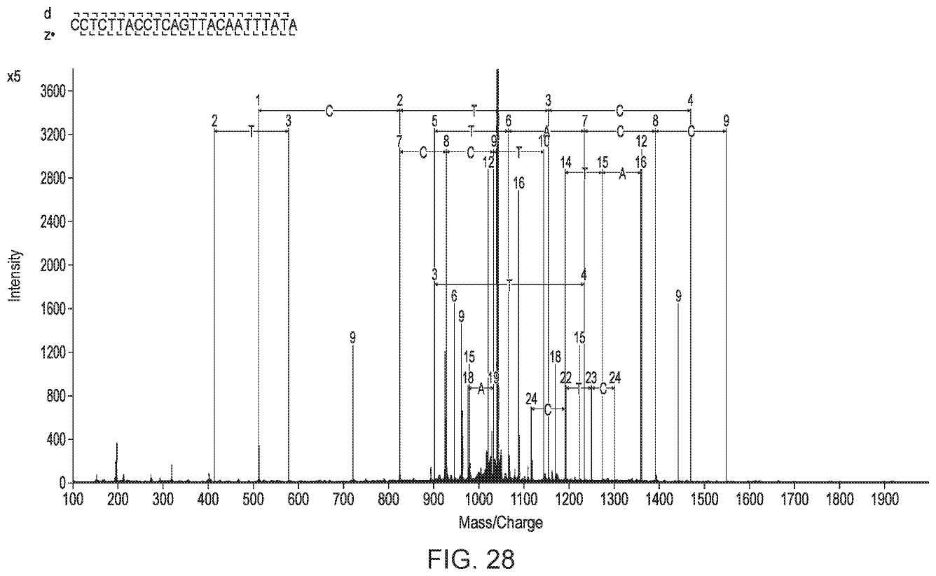

By way of example, FIG. 28 shows a mass spectrum of product ions generated via CID dissociation of a charge reduced species (8−) generated via charge reduction of a precursor ion (9+). ECD at a Ke=0 eV was performed, and the charge reduced species were introduced into the ion trap and the CRS with a charge state of 8− was extracted from the ion trap. The CE energy was 20 eV.

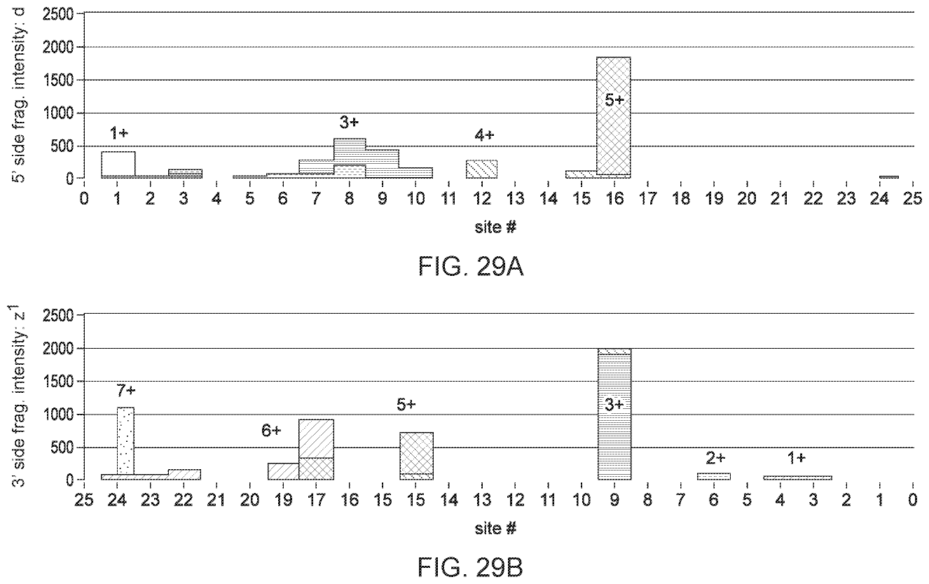

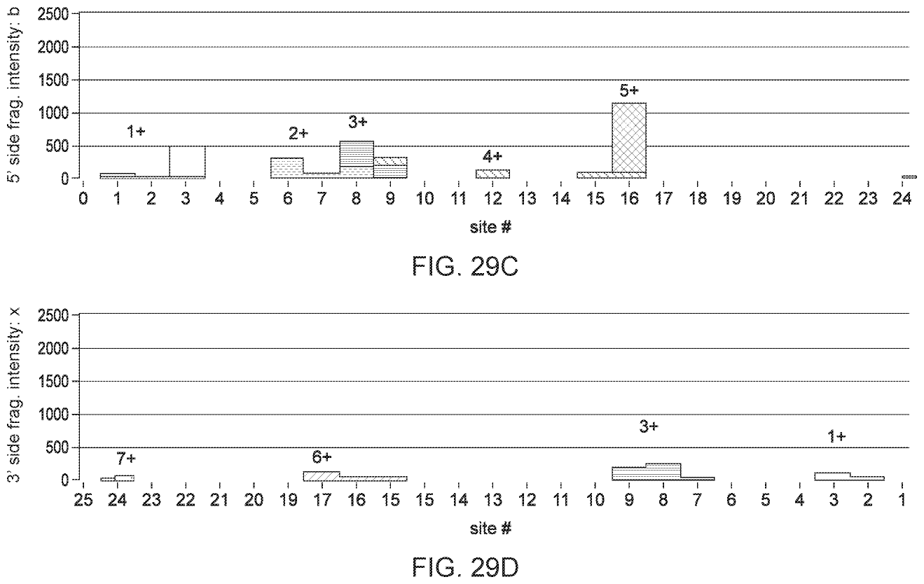

FIGS. 29A, 29B, 29C, and 29D show intensity profiles of d ions (29A), z⋅ ions (29B), b ions (29C) and x ions (29D) in ECD of a morpholino oligomer. Post activation of the charge reduced species produces d and z⋅ ions similar to non-post activated ECD but also produces b and x ions. Charge states are indicated in the graphs of FIGS. 29A, 29B, 29C, and 29D.

The generated product ions were similar to those generated by ECD. At high collision energy (CE=30 eV), the 1 Da ladder appeared. The CID post activation can be applied to ECD products and also to isolated charge species, with the ED performed, e.g., at a Ke=0-3 eV and a CID activation energy in a range of 20±5 eV.

In some embodiments, CID can be substituted by infrared laser-based post-activation (IRMPD) for application to ECD product and/or charge reduced species. By way of example, the IR laser light can be provided by a CO₂ laser.

### Example 2

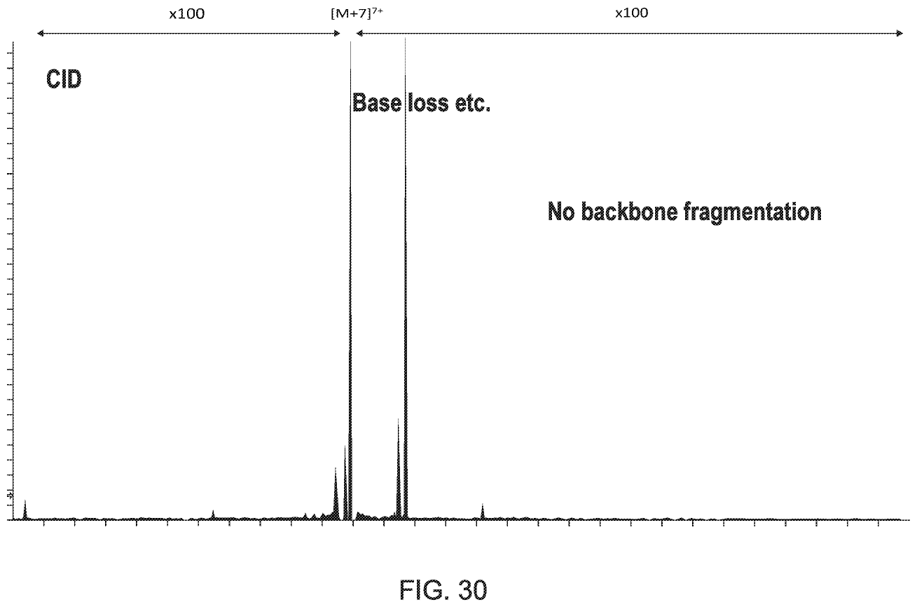

A morpholino oligomer with the following nucleotide sequence was subjected to CID: CCTCTTACCTAGTTAAATTTATA. The mass spectra data depicted in FIG. 30 shows that CID does not produce backbone fragments from the morpholino precursor ions, obviating the need to remove precursor ions before post-activation. Post-activation can be applied to both precursor ions and CRS1 (i.e., CRS generated by capturing one electron).

A morpholino precursor ion at a charge state of +7 was subjected to ECD at electron kinetic energy of 1 eV. The ECD did not result in the generation of any CRS2 (i.e., CRS generated via capture of two electrons). A collisional post-activation at a collision energy of 29 eV was performed by trapping the CRS ions in an ion trap and the target ions into a downstream collision cell Q2 shown in FIG. 28.

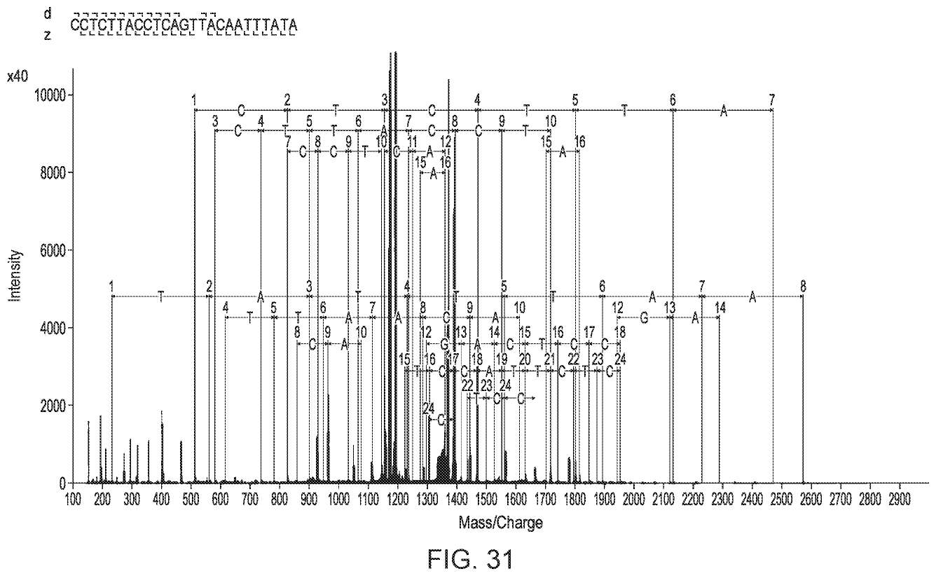

FIG. 31 shows the mass spectrum of the resultant product ions, i.e., the product ions generated via CID dissociation of the charge reduced species.

In this example, the sequence coverage was not as good as that that can be acquired using standard ECD at a high electron kinetic energy. In particular, many small fragments were observed.

The above data shows that many embodiments, CID-based post-activation can be applied to the entire ECD products, e.g., for ECD performed at Ke=0-3 eV. The CID activation energy can be, for example, in a range of about 20±5 eV.

It is also noted that IR laser-based post-activation (IRMPD), e.g., performed via a CO2 laser, can also be applied to the entire ECD products.

### Summary of the Above Observations

In the above examples, ECD produced backbone fragment ions with the main fragmentation channel being d and z⋅ ions and the sub fragmentation channel being b and x ions. Minor dissociation channels leading to N-Me2 loss and the generation of z-H and b-H ions were also observed. 1 Da ladder background was produced at high electron kinetic energy and long electron beam irradiation. Helium did not help reduce the 1 Da ladder background and secondary dissociation. The data also shows that performing ECD at a a Ke=5 eV can lead to purer backbone fragments, i.e., d and z⋅ ions were dominant with less b and x ions, though CRSn (CRS produced via capture of n electrons) can be strong. Further, the data shows that ECD at Ke=15 eV can provide higher sensitivity than lower energies (e.g., 5 eV) and hence can be a useful tool for sensitive applications. 1 Da ladder may appear but the informative fragment ions are much stronger than the background.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

What is claimed is:

1. A mass spectrometric method for sequencing a morpholino oligomer, comprising:
ionizing the morpholino oligomer to generate a positively-charged precursor ion,
dissociating said positively-charged precursor ion using electron-based dissociation to generate a plurality of product ions, and
determining one or more nucleotides of the morpholino oligomer based on analysis of m/z ratios of one or more of the product ions.

2. The mass spectrometric method of claim 1, further comprising generating a mass spectrum of the product ions for determining said m/z ratios.

3. The mass spectrometric method of claim 1, wherein at least a portion of the product ions comprises backbone fragments of the precursor ion.

4. The mass spectrometric method of claim 1, wherein the electron-based dissociation is performed using electrons having an electron kinetic energy in a range of about 10 eV to about 20 eV.

5. The mass spectrometric method of claim 1, wherein the electron-based dissociation is performed using electrons having an electron kinetic energy in a range of about 3 eV to about 7 eV.

6. The mass spectrometric method of claim 1, wherein said step of ionizing the morpholino oligomer comprises using electrospray ionization.

7. The mass spectrometric method of claim 1, further comprising using m/z ratios of d and z product ions to determine the one or more nucleotides of the morpholino oligomer.

8. The mass spectrometric method of claim 1, further comprising using m/z ratios of b and x product ions to determine the one or more nucleotides of the morpholino oligomer.

9. The mass spectrometric method of claim 1, further comprising dissociating at least a portion of the product ions to generate another set of product ions, and wherein optionally the step of dissociating at least a portion of the product ions comprises using collision-induced dissociation (CID) or using infrared multiple photo dissociation (IRMPD), and wherein the IRMPD is performed using a CO₂ laser.

10. A mass spectrometric method for sequencing a morpholino oligomer, comprising:
ionizing a plurality of morpholino oligomers to generate a plurality of positively-charged precursor ions,
dissociating at least a first one of said positively-charged precursor ions using electron capture dissociation at an electron kinetic energy in a range of about 10 eV to about 20 eV to generate a first set of product ions,
dissociating at least a second one of said positively-charged precursor ions using electron capture dissociation at an electron kinetic energy in a range of about 3 eV to about 7 eV to generate a second set of product ions, and
determining a nucleotide sequence of the morpholino oligomer based on analysis of m/z ratios of one or more of the first set of the product ions and one or more of the second set of the product ions.

11. The mass spectrometric method of claim 10, further comprising generating mass spectra of the first and the second set of the product ions to determine said m/z ratios.

12. A mass spectrometric method for sequencing a morpholino oligomer, comprising:
using a processor to compute at least one mass spectrum of product ions generated via electron-based dissociation of a protonated morpholino oligomer, and
determining one or more nucleotides of the morpholino oligomer based on said at least one mass spectrum of the product ions.

13. The mass spectrometric method of claim 12, wherein said electron-based dissociation is performed using electrons having a kinetic energy in a range of about 10 eV to about 20 eV.

14. The mass spectrometric method of claim 12, wherein said electron-based dissociation is performed using electrons having a kinetic energy in a range of about 3 eV to about 7 e V.

15. The mass spectrometric method of claim 12, wherein the step of determining one or more nucleotides of the morpholino oligomer comprises determining a nucleotide sequence of the morpholino oligomer.

16. The mass spectrometric method of claim 12, wherein said at least one mass spectrum of the product ions comprises at least two mass spectra, wherein one of the mass spectra is associated with product ions generated at an electron kinetic energy in a range of about 10 eV to about 20 eV and another one of the mass spectra is associated with product ions generated at an electron kinetic energy in range of about 3 eV to about 7 eV.

17-20. (canceled)
